(19) United States
(12) Patent Application Publication
(10) Pub. No.: US 2006/0116407 A1
(43) Pub. Date: Jun. 1, 2006

(54) Amide derivatives

(76) Inventors: Birgit Bossenmaier, Seefeld (DE); Walter-Gunar Friebe, Mannheim (DE); Eike Hoffmann, Seefeld (DE); Thomas von Hirschheydt, Penzberg (DE); Edgar Voss, Bichl (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/274,497

(22) Filed: Nov. 15, 2005

(30) Foreign Application Priority Data
Nov. 22, 2004 (EP) 04 027 654.5

(51) Int. Cl. A61K 31/427; A61K 31/422; C07D 417/02; C07D 413/02

(52) U.S. Cl. 514/370; 514/375; 548/233; 548/190

(57) ABSTRACT

The present invention relates to the compounds of formula I 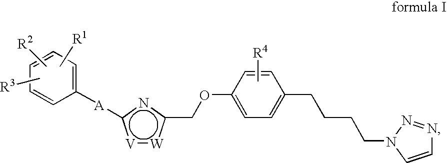 their pharmaceutically acceptable salts, enantiomeric forms, diastereoisomers and racemates, the preparation of the above-mentioned compounds, pharmaceutical compositions containing them and their manufacture, as well as the use of the above-mentioned compounds in the control or prevention of illnesses such as cancer.

### PRIORITY TO RELATED APPLICATIONS

This application claims the benefit of European Application No. 04027654.5, filed Nov. 22, 2004, which is hereby incorporated by reference in its entirety.

### FIELD OF THE INVENTION

The present invention relates to novel amide derivatives, to a process for their manufacture, pharmaceutical compositions containing them and their manufacture as well as the use of these compounds as pharmaceutically active agents.

### BACKGROUND OF THE INVENTION

Protein tyrosine kinases (PTKs) catalyze the phosphorylation of tyrosyl residues in various proteins involved in the regulation of cell growth and differentiation (Wilks, A. F., Progress in Growth Factor Research 2 (1990) 97-111; Chan, A. C., and Shaw, A. S., Curr. Opin. Immunol. 8 (1996) 394-401). Such PTKs can be divided into receptor tyrosine kinases (e.g. EGFR/HER-1, c-erB2/HER-2, c-met, PDGFr, FGFr) and non-receptor tyrosine kinases (e.g. src, Ick). It is known that many oncogenes encode proteins which are aberrant tyrosine kinases capable of causing cell transformation (Yarden, Y., and Ullrich, A., Annu. Rev. Biochem. 57 (1988) 443-478; Larsen et al., Ann. Reports in Med. Chem., 1989, Chpt. 13). Also over-expression of a normal proto-oncogenic tyrosine kinase may result in proliferative disorders.

It is known that receptor tyrosine kinases of the HER-family like HER-2 and EGFR (HER-1) are frequently aberrantly expressed in common cancers such as breast cancer, gastrointestinal cancer (such as colon, rectal or stomach cancer), leukemia and ovarian, bronchial and pancreatic cancer. High levels of these receptors correlate with poor prognosis and response to treatment (Wright, C., et al., Br. J. Cancer 65 (1992) 118-121).

Accordingly, it has been recognized that inhibitors of receptor tyrosine kinases are useful as selective inhibitors of the growth of mammalian cancer cells. Therefore several small molecule compounds as well as monoclonal antibodies are in clinical trials for the treatment of various types of cancer (Baselga, J., and Hammond, L. A., Oncology 63 (Suppl. 1) (2002) 6-16; Ranson, M., and Sliwkowski, M. X., Oncology 63 (suppl. 1) (2002) 17-24).

Some substituted oxazoles are known in the art. WO 98/03505, EP 1 270 571, WO 01/77107, WO 03/031442 and WO 03/059907 disclose related heterocyclic compounds as—tyrosine kinase inhibitors.

However there remains a need for new compounds with improved therapeutic properties, such as enhanced activity, decreased toxicity, better solubility and improved pharmacokinetic profile, to name only a few.

### SUMMARY OF THE INVENTION

The present invention relates to compounds of general formula I and pharmaceutically acceptable salts thereof wherein formula I is: 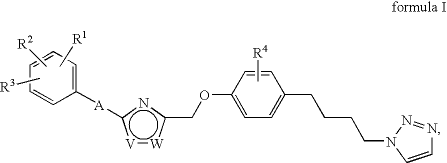
wherein:
R¹ and R² are adjacent and together with the carbon atoms of the phenyl ring to which they are attached form a 5 or 6 membered heterocyclic ring; or alternatively R² is hydrogen and R¹ is selected from the group consisting of:
   (a) hydrogen,
   (b) halogen,
   (c) nitro,
   (d) —SF₅,
   (e) —O-alkyl, wherein the alkyl group is optionally substituted with one or more halogens,
   (f) —S(O)ₙ-alkyl, wherein n is 0, 1 or 2 and wherein the alkyl group is optionally substituted with one or more halogens;
   (g) —S(O)₂NH₂,
   (h) —S(O)₂NH-acyl,
   (i) —S(O)₂NH-heteroaryl,
   (j) —NH-alkyl, wherein the alkyl group is optionally substituted with one or more halogens, and
   (k) alkyl which is optionally substituted with one or more halogens;
R³ is hydrogen, halogen or nitro;
R⁴ is hydrogen or alkyl;
A is —NHC(O)—, —C(O)NH—, —N(alkyl)C(O)— or —C(O)N(alkyl)-; and
V is —S— and W is —CH—, or alternatively V is —CH— and W is —S— or —O—.

The compounds of formula I are useful for preventing or treating proliferative diseases and conditions such as tumor growth and cancer including, but not limited to, breast cancer, leukemia, ovarian cancer, bronchial or lung cancer, pancreatic cancer, and gastrointestinal cancer such as colon cancer, rectal cancer, and stomach cancer.

The compounds of the present invention show activity as inhibitors of the HER-signaling pathway and therefore possess anti-proliferative activity. The present invention provides the compounds of formula I and their pharmaceutically acceptable salts, enantiomeric forms, diastereoisomers and racemates, the preparation of the above-mentioned compounds, compositions containing them and their manufacture as well as the use of the above-mentioned compounds in the control or prevention of illnesses, especially of illnesses and disorders as mentioned above like common human cancers (e.g. breast cancer, gastrointestinal cancer (colon, rectal or stomach cancer), leukemia and ovarian, bronchial and pancreatic cancer) or in the manufacture of corresponding pharmaceutical compositions.

### DETAILED DESCRIPTION OF THE INVENTION

As used herein, the term “alkyl” means a saturated, straight-chain or branched-chain hydrocarbon containing from 1 to 4, preferably from 1 to 2, carbon atoms. Examples are methyl, ethyl, n-propyl, isopropyl, n-butyl, 2-butyl, and t-butyl. If said alkyl group is substituted one or several times by halogen, it is preferably substituted one to five times and more preferably substituted one to three times by halogen; preferably with fluorine or chlorine, and more preferably with fluorine. Examples are difluoromethyl, trifluoromethyl, 2,2,2-trifluoroethyl, perfluorethyl and the like, preferably trifluoromethyl.

As used herein, the term “halogen” means fluorine, chlorine or bromine, preferably fluorine or chlorine.

As used herein, the term “acyl” means a C₂-C₄-, preferably a C₂-C₃-, acyl group such as acetyl, propionyl, butyryl or isobutyryl.

As used herein, the term “heteroaryl” means an unsaturated cyclic hydrocarbon with 5 or 6 ring atoms, preferably 5 ring atoms, of which 1, 2 or 3 atoms are replaced by heteroatoms selected from the group consisting of O, N and S. Such a ring can be substituted, where appropriate, one or two times, preferably one time, by C₁-C₄-alkyl, preferably by C₁-C₂-alkyl. Examples of such rings are thiazole, oxazole, isoxazole, thiadiazole, triazole and the like; preferably thiazole, isoxazole, or thiadiazole.

As used herein the heterocyclic ring formed by R¹ and R² means a saturated or unsaturated cyclic hydrocarbon with 5 or 6 ring atoms of which 1 or 2 atoms are replaced by heteroatoms selected from the group consisting of S, N and O, preferably selected from the group consisting of N and O, and the remaining carbon-atoms, where possible, being optionally once or several times substituted with halogen, preferably fluorine. Preferably said “5 or 6 membered heterocyclic ring” is formed by R¹ and R² being located on two adjacent carbon-atoms of the phenyl ring to which they are attached. Examples of a “5 or 6 membered heterocyclic ring,” including the phenyl ring to which it is attached, are benzo[1,3]dioxole, 2,2-difluoro-benzo[1,3]dioxole, 1H-benzimidazole, 2,3-dihydro-benzo[1,4]dioxine, 3,4-dihydro-2H-benzo[1,4]oxazine and the like; preferablybenzo[1,3]dioxole or 2,2-difluoro-benzo[1,3]dioxole.

Preferred substituents in the definition of R¹ are trifluoromethyl, pentafluorosulfanyl, trifluoromethylsulfanyl, methoxy, difluoromethoxy, trifluoromethoxy, chloro and fluoro, especially trifluoromethoxy, trifluoromethyl and chlorine. A preferred position of the substituent R¹ on the phenyl ring to which it is attached, is para to the group A.

When “R¹ and R² together with the carbon atoms to which they are attached form a 5 or 6 membered heterocyclic ring”, the resulting bicyclic ring system, including the phenyl ring to which R¹ and R² are attached is preferably a 2,2-difluoro-benzo[1,3]dioxolyl or a benzo[1,3]dioxolyl moiety.

The preferred substituent in the definition of R² is hydrogen.

Preferred substituents in the definition of R³ are hydrogen, fluoro and chloro, especially hydrogen and fluoro. A preferred position of R³ on the phenyl ring to which it is attached, is ortho to the group A.

If R⁴ is alkyl, the preferred position of R⁴ on the phenyl ring to which it is attached is meta to the oxygen of the phenolic ether.

As used herein, when referring to the receptor tyrosine kinases of the HER-family like HER-2 and EGFR (HER-1), the acronym “HER” refers to human epidermal receptor and the acronym “EGFR” refers to epidermal growth factor receptor.

As used herein, in relation to mass spectrometry (MS) the term “ESI+” refers to positive electrospray ionization mode and the term “API+” refers to positive atmospheric pressure ionization mode.

As used herein, “DMSO” refers to N,N-dimethylsulfoxide.

As used herein, the term “DMF” refers to N,N-dimethyl formamide.

As used herein, in relation to nuclear magnetic resonance (NMR) the term “D₆-DMSO” refers to deuterated N,N-dimethylsulfoxide.

As used herein, the term “a therapeutically effective amount” of a compound means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration, it may be given as continuous infusion.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

The compounds according to the present invention may exist in the form of their pharmaceutically acceptable salts. The term “pharmaceutically acceptable salt” refers to conventional acid-addition salts that retain the biological effectiveness and properties of the compounds of formula I and are formed from suitable non-toxic organic or inorganic acids. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, naphthalenesulfonic acid, naphthalenedisulfonic acid, methanesulfonic acid, ethanesulfonic acid, salicylic acid, oxalic acid, succinic acid, citric acid, malic acid, lactic acid, fumaric acid, and the like. The chemical modification of a pharmaceutical compound (i.e. a drug) into a salt is a technique well known to pharmaceutical chemists to obtain improved physical and chemical stability, hygroscopicity, flowability and solubility of compounds. See, e.g., Stahl, P. H., and Wermuth, G., (editors), Handbook of Pharmaceutical Salts, Verlag Helvetica Chimica Acta (VHCA), Zürich, (2002) or Bastin, R. J., et al., Organic Proc. Res. Dev. 4 (2000) 427-435.

Preferred are the pharmaceutically acceptable salts, which are formed with p-toluenesulfonic acid, naphthalenesulfonic acid, naphthalenedisulfonic acid, methanesulfonic acid and hydrochloric acid.

In one embodiment of the invention, R⁴ of formula I is hydrogen.

In another embodiment of the invention are the compounds according to formula I, wherein:
R¹ is selected from the group consisting of:
   (a) chlorine,
   (b) —O-alkyl, wherein the alkyl group is optionally substituted with one or more fluorines;
   (c) —S-alkyl, wherein the alkyl group is optionally substituted with one or more fluorines; and
   (d) alkyl, which is optionally substituted with one or more fluorines;
R² is hydrogen; and
R³ is hydrogen or fluorine.

Another embodiment of the invention are the compounds according to formula I, wherein:
R¹ is selected from the group consisting of:
   (a) chlorine,
   (b)-O-alkyl, wherein the alkyl group is optionally substituted with one or more fluorines;
   (c) —S-alkyl, wherein the alkyl group is optionally substituted with one or more fluorines; and
   (d) alkyl, which is optionally substituted with one or more fluorines;
R² is hydrogen;
R³ is hydrogen or fluorine; and
R⁴ is hydrogen.

Another embodiment of the invention are the compounds according to formula I, wherein:
A is —NHC(O)— or —N(alkyl)C(O)—.

An embodiment of the invention are the compounds according to formula I, wherein:
R⁴ is hydrogen; and
A is —NHC(O)— or —N(alkyl)C(O)—.

Another embodiment of the invention are the compounds according to formula I, wherein:
R¹ is selected from the group consisting of:
   (a) chlorine;
   (b) —O-alkyl, wherein the alkyl group is optionally substituted with one or more fluorines;
   (c) —S-alkyl, wherein the alkyl group is optionally substituted with one or more fluorines; and
   (d) alkyl, which is optionally substituted with one or more fluorines;
R² is hydrogen;
R³ is hydrogen or fluorine; and
A is —NHC(O)— or —N(alkyl)C(O)—.

Another embodiment of the invention are the compounds according to formula I, wherein:
R¹ is selected from the group consisting of:
   (a) chlorine;
   (b) —O-alkyl, wherein the alkyl group is optionally substituted with one or more fluorines;
   (c) —S-alkyl, wherein the alkyl group is optionally substituted with one or more fluorines; and
   (d) alkyl, which is optionally substituted with one or more fluorines;
R² is hydrogen;
R³ is hydrogen or fluorine;
R⁴ is hydrogen; and
A is —NHC(O)— or —N(alkyl)C(O)—.

Another embodiment of the invention are the compounds according to formula I, wherein:
A is —NHC(O)— or —N(alkyl)C(O)—;
V is —CH—; and
W is —S— or —O—.

Another embodiment of the invention are the compounds according to formula I, wherein:
R⁴ is hydrogen;
A is —NHC(O)— or —N(alkyl)C(O)—;
V is —CH—; and
W is —S— or —O—.

Another embodiment of the invention are the compounds according to formula I, wherein:
R¹ is selected from the group consisting of:
   (a) chlorine;
   (b) —O-alkyl, wherein the alkyl group is optionally substituted with one or more fluorines;
   (c) —S-alkyl, wherein the alkyl group is optionally substituted with one or more fluorines; and
   (d) alkyl; which is optionally substituted with one or more fluorines;
R² is hydrogen;
R³ is hydrogen or fluorine.
R⁴ is hydrogen;
A is —NHC(O)— or —N(alkyl)C(O)—;
V is —CH—; and
W is —S— or —O—.

Another embodiment of the invention are the compounds according to formula I, wherein:
R¹ is selected from the group consisting of:
   (a) chlorine;
   (b) —O—CF₃;
   (c) —O—CF₂;
   (d) —S—CF₃;
   (e) —S—CF₂;
   (f) —CF₃; and
   (g) —SF₅;
R² is hydrogen;
R³ is hydrogen, fluorine, chlorine or nitro;
R⁴ is hydrogen;
A is —NHC(O)— or —N(alkyl)C(O)—;
V is —CH—; and
W is —S— or —O—.

Such compounds are for example:
2-[4-(4-[1,2,3]Triazol-1-yl-butyl)-phenoxymethyl]-oxazole-4-carboxylic acid (4-chloro-phenyl)-methyl-amide;
2-[4-(4-[1,2,3]Triazol-1-yl-butyl)-phenoxymethyl]-oxazole-4-carboxylic acid (2-nitro-4-trifluoromethyl-phenyl)-amide;
2-[4-(4-[1,2,3]Triazol-1-yl-butyl)-phenoxymethyl]-oxazole-4-carboxylic acid (4-chloro-phenyl)-amide;
2-[4-(4-[1,2,3]Triazol-1-yl-butyl)-phenoxymethyl]-oxazole-4-carboxylic acid (4-difluoromethylsulfanyl-phenyl)-amide;
2-[4-(4-[1,2,3]Triazol-1-yl-butyl)-phenoxymethyl]-oxazole-4-carboxylic acid (4-trifluoro-methoxy-phenyl)-amide;
2-[4-(4-[1,2,3]Triazol-1-yl-butyl)-phenoxymethyl]-oxazole-4-carboxylic acid (4-trifluoro-methylsulfanyl-phenyl)-amide;
2-[4-(4-[1,2,3]Triazol-1-yl-butyl)-phenoxymethyl]-oxazole-4-carboxylic acid (3-chloro-4-fluoro-phenyl)-amide;
2-[4-(4-[1,2,3]Triazol-1-yl-butyl)-phenoxymethyl]-oxazole-4-carboxylic acid (4-trifluoromethyl-phenyl)-amide;
2-[4-(4-[1,2,3]Triazol-1-yl-butyl)-phenoxymethyl]-oxazole-4-carboxylic acid (4-chloro-2-fluoro-phenyl)-amide;
2-[4-(4-[1,2,3]Triazol-1-yl-butyl)-phenoxymethyl]-oxazole-4-carboxylic acid (4-difluoromethoxy-phenyl)-amide;
2-[4-(4-[1,2,3]Triazol-1-yl-butyl)-phenoxymethyl]-oxazole-4-carboxylic acid (3-trifluoromethyl-phenyl)-amide;
2-[4-(4-[1,2,3]Triazol-1-yl-butyl)-phenoxymethyl]-oxazole-4-carboxylic acid (4-chloro-3-trifluoromethyl-phenyl)-amide;
2-[4-(4-[1,2,3]Triazol-1-yl-butyl)-phenoxymethyl]-oxazole-4-carboxylic acid methyl-(4-trifluoromethoxy-phenyl)-amide;
2-[4-(4-[1,2,3]Triazol-1-yl-butyl)-phenoxymethyl]-oxazole-4-carboxylic acid (4-nitro-2-trifluoromethyl-phenyl)-amide;
2-[4-(4-[1,2,3]Triazol-1-yl-butyl)-phenoxymethyl]-thiazole-4-carboxylic acid (4-trifluoromethyl-phenyl)-amide;
2-[4-(4-[1,2,3]Triazol-1-yl-butyl)-phenoxymethyl]-thiazole-4-carboxylic acid (4-chloro-phenyl)-amide;
2-[4-(4-[1,2,3]Triazol-1-yl-butyl)-phenoxymethyl]-thiazole-4-carboxylic acid (4-trifluoromethoxy-phenyl)-amide;
2-[4-(4-[1,2,3]Triazol-1-yl-butyl)-phenoxymethyl]-oxazole-4-carboxylic acid (2,6-dichloro-phenyl)-amide; and
2-[4-(4-[1,2,3]Triazol-1-yl-butyl)-phenoxymethyl]-oxazole-4-carboxylic acid (2,4-dichloro-phenyl)-amide.

Another embodiment of the invention are the compounds according to formula I, wherein:
R¹ and R² are adjacent and together with the carbon atoms of the phenyl ring to which they are attached form a 2,2-difluoro-benzo[1,3]dioxolyl moiety or a benzo[1,3]dioxolyl moiety; or alternatively R² is hydrogen and R¹ is selected from the group consisting of:
   (a) hydrogen,
   (b) fluorine,
   (c) bromine,
   (d) —O-alkyl,
   (e) —S-alkyl,
   (f) alkyl,
   (g) —S(O)₂NH₂,
   (h) —S(O)₂NH-acyl, and
   (i) —S(O)₂NH-heteroaryl;
R³ is hydrogen, fluorine or nitro;
R⁴ is hydrogen;
A is —NHC(O)— or —N(alkyl)C(O)—;
V is —CH—; and
W is —S—, or —O—.

Such compounds are for example:
2-[4-(4-[1,2,3]Triazol-1-yl-butyl)-phenoxymethyl]-oxazole-4-carboxylic acid benzo[1,3]dioxol-5-yl-amide;
2-[4-(4-[1,2,3]Triazol-1-yl-butyl)-phenoxymethyl]-oxazole-4-carboxylic acid (3,5-difluoro-phenyl)-amide;
2-[4-(4-[1,2,3]Triazol-1-yl-butyl)-phenoxymethyl]-oxazole-4-carboxylic acid (2,4-difluoro-phenyl)-amide;
2-[4-(4-[1,2,3]Triazol-1-yl-butyl)-phenoxymethyl]-oxazole-4-carboxylic acid (2,2-difluoro-benzo [1,3] dioxol-5-yl)-amide;
2-[4-(4-[1,2,3]Triazol-1-yl-butyl)-phenoxymethyl]-oxazole-4-carboxylic acid (4-(thiazol-2-ylsulfamoyl)-phenyl)-amide;
2-[4-(4-[1,2,3]Triazol-1-yl-butyl)-phenoxymethyl]-oxazole-4-carboxylic acid [4-(5-methyl-[1,3,4] thiadiazol-2-ylsulfamoyl)-phenyl]-amide;
2-[4-(4-[1,2,3]Triazol-1-yl-butyl)-phenoxymethyl]-oxazole-4-carboxylic acid [4-(5-methyl-isoxazol-3-ylsulfamoyl)-phenyl]-amide;
2-[4-(4-[1,2,3]Triazol-1-yl-butyl)-phenoxymethyl]-oxazole-4-carboxylic acid (4-acetylsulfamoyl-phenyl)-amide;
2-[4-(4-[1,2,3]Triazol-1-yl-butyl)-phenoxymethyl]-oxazole-4-carboxylic acid (4-bromo-2-fluoro-phenyl)-amide;
2-[4-(4-[1,2,3]Triazol-1-yl-butyl)-phenoxymethyl]-oxazole-4-carboxylic acid (3-fluoro-2-methyl-phenyl)-amide;
2-[4-(4-[1,2,3]Triazol-1-yl-butyl)-phenoxymethyl]-oxazole-4-carboxylic acid (4-fluoro-3-nitro-phenyl)-amide;
2-[4-(4-[1,2,3]Triazol-1-yl-butyl)-phenoxymethyl]-oxazole-4-carboxylic acid (4-methylsulfanyl-phenyl)-amide;
2-[4-(4-[1,2,3]Triazol-1-yl-butyl)-phenoxymethyl]-oxazole-4-carboxylic acid (4-fluoro-2-methyl-phenyl)-amide;
2-[4-(4-[1,2,3]Triazol-1-yl-butyl)-phenoxymethyl]-oxazole-4-carboxylic acid (4-tert-butyl-phenyl)-amide;
2-[4-(4-[1,2,3]Triazol-1-yl-butyl)-phenoxymethyl]-oxazole-4-carboxylic acid p-tolylamide;
2-[4-(4-[1,2,3]Triazol-1-yl-butyl)-phenoxymethyl]-oxazole-4-carboxylic acid methyl-phenyl-amide;
2-[4-(4-[1,2,3]Triazol-1-yl-butyl)-phenoxymethyl]-oxazole-4-carboxylic acid (4-fluoro-phenyl)-amide;
2-[4-(4-[1,2,3]Triazol-1-yl-butyl)-phenoxymethyl]-oxazole-4-carboxylic acid (4-fluoro-3-methyl-phenyl)-amide;
2-[4-(4-[1,2,3]Triazol-1-yl-butyl)-phenoxymethyl]-oxazole-4-carboxylic acid (2-fluoro-phenyl)-amide;
2-[4-(4-[1,2,3]Triazol-1-yl-butyl)-phenoxymethyl]-oxazole-4-carboxylic acid (3-fluoro-phenyl)-amide;
2-[4-(4-[1,2,3]Triazol-1-yl-butyl)-phenoxymethyl]-oxazole-4-carboxylic acid (3-fluoro-4-methyl-phenyl)-amide;
2-[4-(4-[1,2,3]Triazol-1-yl-butyl)-phenoxymethyl]-oxazole-4-carboxylic acid (4-methoxy-phenyl)-amide; and
2-[4-(4-[1,2,3]Triazol-1-yl-butyl)-phenoxymethyl]-oxazole-4-carboxylic acid (3,4-difluoro-phenyl)-amide.

Another embodiment of the invention are the compounds according to formula I, wherein:
A is —NHC(O)— or —N(alkyl)C(O)—;
V is —S—; and
W is —CH—.

Another embodiment of the invention are the compounds according to formula I, wherein:
R⁴ is hydrogen;
A is —NHC(O)— or —N(alkyl)C(O)—;
V is —S—; and
W is —CH—.

Another embodiment of the invention are the compounds according to formula I, wherein:
R¹ is selected from the group consisting of:
   (a) chlorine;
   (b) —O-alkyl, wherein the alkyl group is optionally substituted with one or more fluorines;
   (c) —S-alkyl, wherein the alkyl group is optionally substituted with one or more fluorines; and
   (d) alkyl, which is optionally substituted with one or more fluorines;
R² is hydrogen;
R³ is hydrogen or fluorine;
R⁴ is hydrogen;
A is —NHC(O)— or —N(alkyl)C(O)—;
V is —S—; and
W is —CH—.

Another embodiment of the invention are the compounds according to formula I, wherein:
R¹ is selected from the group consisting of:
   (a) chlorine;
   (b) —O-alkyl, wherein the alkyl group is optionally substituted with one or more fluorines;
   (c) —SF₅; and
   (d) alkyl, which is optionally substituted with one or more fluorines;
R² is hydrogen;
R³ is hydrogen or fluorine;
R⁴ is hydrogen;
A is —NHC(O)— or —N(alkyl)C(O)—;
V is —S—; and
W is —CH—.

Such compounds are for example:
4-[4-(4-[1,2,3]Triazol-1-yl-butyl)-phenoxymethyl]-thiazole-2-carboxylic acid (4-chloro-phenyl)-amide;
4-[4-(4-[1,2,3]Triazol-1-yl-butyl)-phenoxymethyl]-thiazole-2-carboxylic acid (4-trifluoromethoxy-phenyl)-amide; and
4-[4-(4-[1,2,3]Triazol-1-yl-butyl)-phenoxymethyl]-thiazole-2-carboxylic acid (4-pentafluorosulfanyl-phenyl)-amide.

Another embodiment of the invention are the compounds according to formula I, wherein:
A is —C(O)NH— or —C(O)N(alkyl)-.

An embodiment of the invention are the compounds according to formula I, wherein:
R⁴ is hydrogen; and
A is —C(O)NH— or —C(O)N(alkyl)-.

Another embodiment of the invention are the compounds according to formula I, wherein:
R¹ is selected from the group consisting of:
   (a) chlorine;
   (b) —O-alkyl, wherein the alkyl group is optionally substituted with one or more fluorines;
   (c) —S-alkyl, wherein the alkyl group is optionally substituted with one or more fluorines; and
   (d) alkyl, which is optionally substituted with one or more fluorines;
R² is hydrogen;
R³ is hydrogen or fluorine; and
A is —C(O)NH— or —C(O)N(alkyl)-.

Another embodiment of the invention are the compounds according to formula I, wherein:
R¹ is selected from the group consisting of:
   (a) chlorine;
   (b) —O-alkyl, wherein the alkyl group is optionally substituted with one or more fluorines;
   (c) —S-alkyl, wherein the alkyl group is optionally substituted with one or more fluorines; and
   (d) alkyl, which is optionally substituted with one or more fluorines;
R² is hydrogen;
R³ is hydrogen or fluorine;
R⁴ is hydrogen; and
A is —C(O)NH— or —C(O)N(alkyl)-.

Another embodiment of the invention are the compounds according to formula I, wherein:
A is —C(O)NH— or —C(O)N(alkyl)-;
V is —S—; and
W is —CH—.

Another embodiment of the invention are the compounds according to formula I, wherein:
R⁴ is hydrogen;
A is —C(O)NH— or —C(O)N(alkyl)-;
V is —S—; and
W is —CH—.

Another embodiment of the invention are the compounds according to formula I, wherein:
R¹ is selected from the group consisting of:
   (a) chlorine;
   (b) —O-alkyl, wherein the alkyl group is optionally substituted with one or more fluorines;
   (c) —S-alkyl, wherein the alkyl group is optionally substituted with one or more fluorines; and
   (d) alkyl, which is optionally substituted with one or more fluorines;
R² is hydrogen;
R³ is hydrogen or fluorine;
R⁴ is hydrogen;
A is —C(O)NH— or —C(O)N(alkyl)-;
V is —S—; and
W is —CH—.

Another embodiment of the invention are the compounds according to formula I, wherein:
R¹ is selected from the group consisting of:
   (a) chlorine;
   (b) —O-alkyl, wherein the alkyl group is optionally substituted with one or more fluorines; and
   (c) alkyl, which is optionally substituted with one or more fluorines;
R² is hydrogen;
R³ is hydrogen, chlorine or fluorine;
R⁴ is hydrogen;
A is —C(O)NH— or —C(O)N(alkyl)-;
V is —S—; and
W is —CH—.

Such compounds are for example:
N-{4-[4-(4-[1,2,3]Triazol-1-yl-butyl)-phenoxymethyl]-thiazol-2-yl}-3-trifluoromethyl-benzamide;
N-{4-[4-(4-[1,2,3]Triazol-1-yl-butyl)-phenoxymethyl]-thiazol-2-yl}-4-trifluoromethoxy-benzamide;
4-Chloro-N-{4-[4-(4-[1,2,3]triazol-1-yl-butyl)-phenoxymethyl]-thiazol-2-yl}-benzamide;
4-Chloro-3-fluoro-N-{4-[4-(4-[1,2,3]triazol-1-yl-butyl)-phenoxymethyl]-thiazol-2-yl}-benzamide;
3,4-Dichloro-N-{4-[4-(4-[1,2,3]triazol-1-yl-butyl)-phenoxymethyl]-thiazol-2-yl}-benzamide;
2-Fluoro-N-{4-[4-(4-[1,2,3]triazol-1-yl-butyl)-phenoxymethyl]-thiazol-2-yl}-4-trifluoromethyl-benzamide;
3-Chloro-N-{4-[4-(4-[1,2,3]triazol-1-yl-butyl)-phenoxymethyl]-thiazol-2-yl}-benzamide;
4-Chloro-2-fluoro-N-{4-[4-(4-[1,2,3]triazol-1-yl-butyl)-phenoxymethyl]-thiazol-2-yl}-benzamide;
4-Methyl-N-{4-[4-(4-[1,2,3]triazol-1-yl-butyl)-phenoxymethyl]-thiazol-2-yl}-benzamide;
2-Chloro-N-{4-[4-(4-[1,2,3]triazol-1-yl-butyl)-phenoxymethyl]-thiazol-2-yl}-benzamide;
2-Fluoro-N-{4-[4-(4-[1,2,3]triazol-1-yl-butyl)-phenoxymethyl]-thiazol-2-yl}-6-trifluoromethyl-benzamide;
3-Chloro-4-fluoro-N-{4-[4-(4-[1,2,3]triazol-1-yl-butyl)-phenoxymethyl]-thiazol-2-yl}-benzamide;
N-{4-[4-(4-[1,2,3]Triazol-1-yl-butyl)-phenoxymethyl]-thiazol-2-yl}-4-trifluoromethyl-benzamide;
4-tert-Butyl-N-{4-[4-(4-[1,2,3]triazol-1-yl-butyl)-phenoxymethyl]-thiazol-2-yl}-benzamide; and
4-Difluoromethoxy-N-{4-[4-(4-[1,2,3]triazol-1-yl-butyl)-phenoxymethyl]-thiazol-2-yl}-benzamide.

Another embodiment of the invention are the compounds according to formula I, wherein:
R¹ is selected from the group consisting of:
   (a) nitro;
   (b) cyano;
   (c) —S-alkyl, wherein the alkyl group is optionally substituted with one or more fluorines;
   (d) fluorine; and
   (e) alkyl, which is optionally substituted with one or more fluorines;
R² is hydrogen;
R³ is hydrogen or fluorine;
R⁴ is hydrogen;
A is —C(O)NH— or —C(O)N(alkyl)-;
V is —S—; and
W is —CH—.

Such compounds are for example:
4-Nitro-N-{4-[4-(4-[1,2,3]triazol-1-yl-butyl)-phenoxymethyl]-thiazol-2-yl}-benzamide;
2,4-Difluoro-N-{4-[4-(4-[1,2,3]triazol-1-yl-butyl)-phenoxymethyl]-thiazol-2-yl}-benzamide;
N-{4-[4-(4-[1,2,3]Triazol-1-yl-butyl)-phenoxymethyl]-thiazol-2-yl}-4-trifluoromethylsulfanyl-benzamide;
4-Cyano-N-{4-[4-(4-[1,2,3]triazol-1-yl-butyl)-phenoxymethyl]-thiazol-2-yl}-benzamide;
2,2-Difluoro-benzo [1,3] dioxole-5-carboxylic acid {4-[4-(4-[1,2,3]triazol-1-yl-butyl)-phenoxymethyl]-thiazol-2-yl}-amide;
2,5-Difluoro-N-{4-[4-(4-[1,2,3]triazol-1-yl-butyl)-phenoxymethyl]-thiazol-2-yl}-benzamide;
2,3-Difluoro-N-{4-[4-(4-[1,2,3]triazol-1-yl-butyl)-phenoxymethyl]-thiazol-2-yl}-benzamide;
3,5-Difluoro-N-{4-[4-(4-[1,2,3]triazol-1-yl-butyl)-phenoxymethyl]-thiazol-2-yl}-benzamide;
2-Fluoro-N-{4-[4-(4-[1,2,3]triazol-1-yl-butyl)-phenoxymethyl]-thiazol-2-yl}-benzamide;
3-Fluoro-N-{4-[4-(4-[1,2,3]triazol-1-yl-butyl)-phenoxymethyl]-thiazol-2-yl}-benzamide; and
4-Fluoro-3-methyl-N-{4-[4-(4-[1,2,3]triazol-1-yl-butyl)-phenoxymethyl]-thiazol-2-yl}-benzamide.

Another embodiment of the invention are the compounds according to formula I, wherein:
A is —C(O)NH— or —C(O)N(alkyl)-;
V is —CH—; and
W is —S— or —O—.

Another embodiment of the invention are the compounds according to formula I, wherein:
R⁴ is hydrogen;
A is —C(O)NH— or —C(O)N(alkyl)-;
V is —CH—; and
W is —S— or —O—.

Another embodiment of the invention are the compounds according to formula I, wherein:
R¹ is selected from the group consisting of:
   (a) chlorine;
   (b) —O-alkyl; wherein the alkyl group is optionally substituted with one or more fluorines;
   (c) —S-alkyl, wherein the alkyl group is optionally substituted with one or more fluorines; and
   (d) alkyl, which is optionally substituted with one or more fluorines;
R² is hydrogen;
R³ is hydrogen or fluorine;
R⁴ is hydrogen;
A is —C(O)NH— or —C(O)N(alkyl)-;
V is —CH—; and
W is —S— or —O—.

Another embodiment of the invention are the compounds according to formula I, wherein:
R⁴ is methyl.

Another embodiment of the invention are the compounds according to formula I, wherein:
R¹ is selected from the group consisting of:
   (a) chlorine;
   (b) —O-alkyl, wherein the alkyl group is optionally substituted with one or more fluorines;
   (c) —S-alkyl, wherein the alkyl group is optionally substituted with one or more fluorines; and
   (d) alkyl, which is optionally substituted with one or more fluorines;
R² is hydrogen;
R³ is hydrogen or fluorine;
R⁴ is methyl;
A is —NHC(O)— or —N(alkyl)C(O)—;
V is —CH—; and
W is —S— or —O—.

Another embodiment of the invention are the compounds according to formula I, wherein:
R¹ is selected from the group consisting of:
   (a) chlorine;
   (b)—O—CF₃; and
   (c) —CF₃;
R² is hydrogen;
R³ is hydrogen;
R⁴ is methyl;
A is —NHC(O)— or —N(alkyl)C(O)—;
V is —CH—; and
W is —S— or —O—.

Such compounds are for example:
2-[3-Methyl-4-(4-[1,2,3]triazol-1-yl-butyl)-phenoxymethyl]-oxazole-4-carboxylic acid (4-trifluoromethyl-phenyl)-amide;
2-[3-Methyl-4-(4-[1,2,3]triazol-1-yl-butyl)-phenoxymethyl]-oxazole-4-carboxylic acid (4-chloro-phenyl)-amide;
2-[3-Methyl-4-(4-[1,2,3]triazol-1-yl-butyl)-phenoxymethyl]-oxazole-4-carboxylic acid (3-chloro-phenyl)-amide;
2-[3-Methyl-4-(4-[1,2,3]triazol-1-yl-butyl)-phenoxymethyl]-thiazole-4-carboxylic acid (4-trifluoromethyl-phenyl)-amide;
2-[3-Methyl-4-(4-[1,2,3]triazol-1-yl-butyl)-phenoxymethyl]-thiazole-4-carboxylic acid (4-chloro-phenyl)-amide; and
2-[3-Methyl-4-(4-[1,2,3]triazol-1-yl-butyl)-phenoxymethyl]-thiazole-4-carboxylic acid (3-chloro-phenyl)-amide.

Still another embodiment of the invention is a process for the manufacture of the compounds of formula Ia, wherein:
a) the compound of formula V 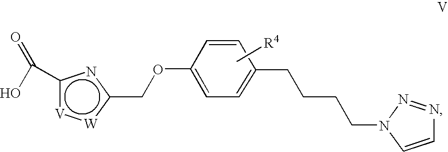
wherein R⁴, V and W have the significance as given in formula I above, is reacted with a compound of formula VI 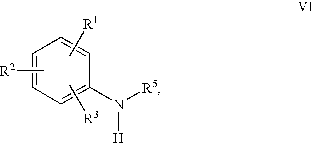
wherein R¹, R² and R³ have the significance as given in formula I above and
wherein R⁵ is hydrogen;
   to give the respective compound of formula Ia; 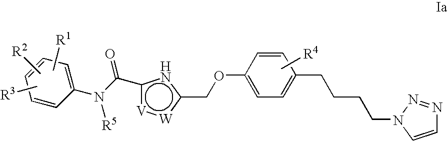
wherein R¹, R², R³ and R⁴ have the significance as given in formula I above and
wherein R⁵ is hydrogen or alkyl;
   b) optionally, said compound of formula Ia is isolated from the reaction mixture, and
   c) optionally, converted into a pharmaceutically acceptable salt.

Still another embodiment of the invention is a process for the manufacture of the compounds of formula Ib, wherein:
a) the compound of formula IX, 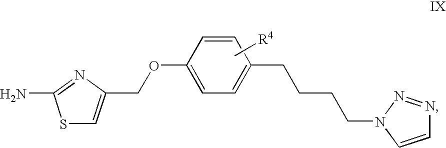
wherein R⁴ has the significance as given in formula I above;
   is reacted with a compound of formula X 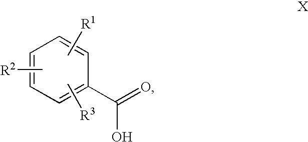
wherein R¹, R², R³ have the significance as given in formula I above, to give the respective compound of formula Ib; 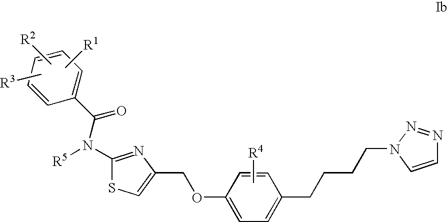
wherein R¹, R², R³ and R⁴ have the significance as given in formula I above and wherein R⁵ is hydrogen;
   b) optionally, the compound of formula Ib obtained in a) is further reacted with a suitable alkyl halide to give the respective compound of formula Ib wherein R⁵ is alkyl;
   c) optionally, said compound of formula Ib is isolated from the reaction mixture, and
   d) optionally, converted into a pharmaceutically acceptable salt.

The amide derivatives of the general formula I, or a pharmaceutically acceptable salt thereof, may be prepared by any process known to be applicable for the preparation of chemically-related compounds by the one skilled in the art. Such processes, when used to prepare the amide derivatives of formula I, or a pharmaceutically-acceptable salt thereof, are provided as a further feature of the invention and are illustrated by the following representative examples of scheme 1, in which, unless otherwise stated, V, W, A, R¹, R², R³ and R⁴ have the significance given herein before. Necessary starting materials may be obtained by standard procedures of organic chemistry. The preparation of such starting materials is described within the accompanying non-limiting examples. Alternatively, necessary starting materials are obtainable by analogous procedures to those illustrated which are within the ordinary skill of an organic chemist.

### Scheme 1

The manufacture of the compounds of formula I varies according to the nature of “A” in formula I. The compounds of the present invention wherein “A” is —NR⁵C(O)— and R⁵ is hydrogen or alkyl can be prepared according to scheme 1, and are named Ia.

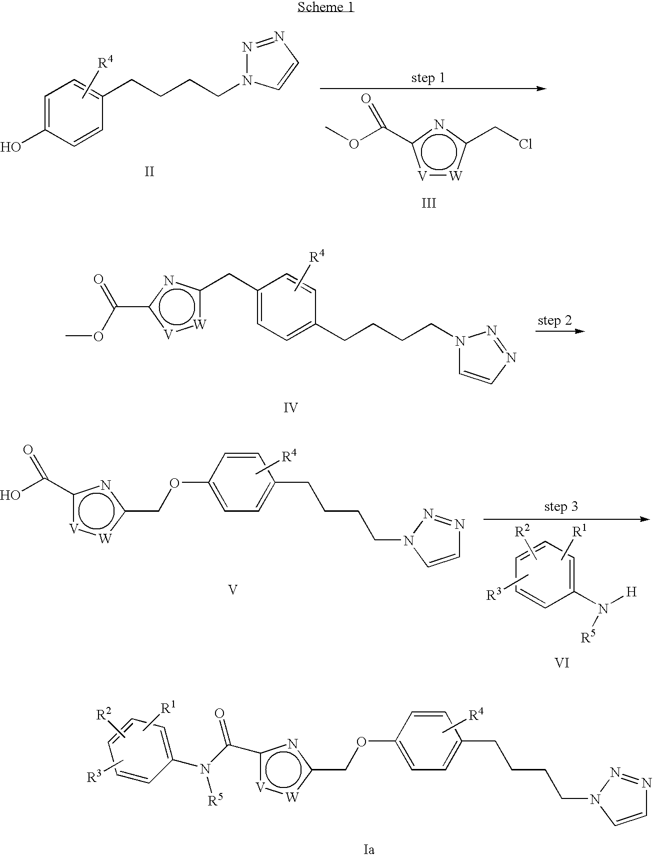

In scheme 1, V, W, R¹, R², R³ and R⁴ have the significance given herein before for formula I and R⁵ is hydrogen or alkyl.

Step 1

In step 1, scheme 1 the compounds of formula II can be obtained by reactions well known to someone skilled in the art) e.g. by alkylation of 4-(4-[1,2,3]Triazol-1-yl-butyl)-phenol with compounds of formula III. Typical bases for this reaction are sodium methylate, sodium hydride, lithium diisopropyl amide and cesium carbonate. The alkylation can be carried out in the presence of potassium iodide or sodium iodide in solvents like methanol, ethanol, isopropanol and N,N-dimethylformamide (DMF). The reaction temperatures may vary from 50° C. to 150° C. Oxazoles or thiazoles of formula III can be synthesized by a commonly known method or a modification thereof. (2-Chlormethyl-oxa/thiazole-4-carboxylic acid methyl ester: Hermitage, S. A., et al., Organic Process Research & Development 5 (2001) 37-44; 4-Chlormethyl-thiazole-2-carboxylic acid ethyl ester: Lee, C. B., et al., J. Am. Chem. Soc. 123 (2001) 5249-5259.)

Step 2

In step 2 the hydrolysis of the esters of formula IV is achieved by standard methods for someone skilled in the art. Typically used bases are e.g. sodium hydroxide (NaOH), potassium hydroxide (KOH), lithium hydroxide (LiOH) in solvents like water, tetrahydrofuran (THF), methanol, ethanol or mixtures thereof at temperature between 0° C. and 150° C., yielding the carboxylic acids of formula V.

Step 3

In step 3 the obtained carboxylic acids of formula V are reacted with anilines of formula VI using standard methods (e.g. Han, S.-Y., and Kim, Y.-A., Tetrahedron 60 (2004) 2447-2467) for someone skilled in the art, e.g. by activating the carboxylic acid group in the compounds of formula V with 1-ethyl-3-(3′-dimethylaminopropyl)carbodiimide (EDCI), N,N′-carbonyl diimidazole (CDI), hydroxybenzotriazole (HOBt) or thionylchloride in solvents like THF, dichloromethane, DMF or mixtures thereof and at temperatures varying from −30° C. to 50° C., yielding derivatives of formula Ia.

Alternatively, the compounds of formula Ia wherein R⁵ is alkyl can be obtained by introducing the R⁵-alkyl group after the last reaction step by alkylation of the corresponding amides of formula Ia (R⁵ is hydrogen). This reaction is typically achieved with alkyl halides such as for example the alkyl halides of the formula R⁵-Hal, wherein “Hal” is a halogen-atom, preferably iodine or bromine and R⁵ is alkyl. The reaction is carried out in the presence of a base like NaOH, KOH, triethyl amine or sodium hydride and in solvents like acetone, ethyl acetate, methanol, ethanol, DMF or mixtures thereof at temperatures varying from 0° C. to 150° C.

Furthermore the sequence of the reaction steps can vary.

Scheme 2:

The manufacture of the compounds of formula I varies according to the nature of “A” in formula I. The compounds of the present invention wherein “A” is —O—, and V is —S— and W is —CH— can be prepared according to scheme 1, and are named Ib.

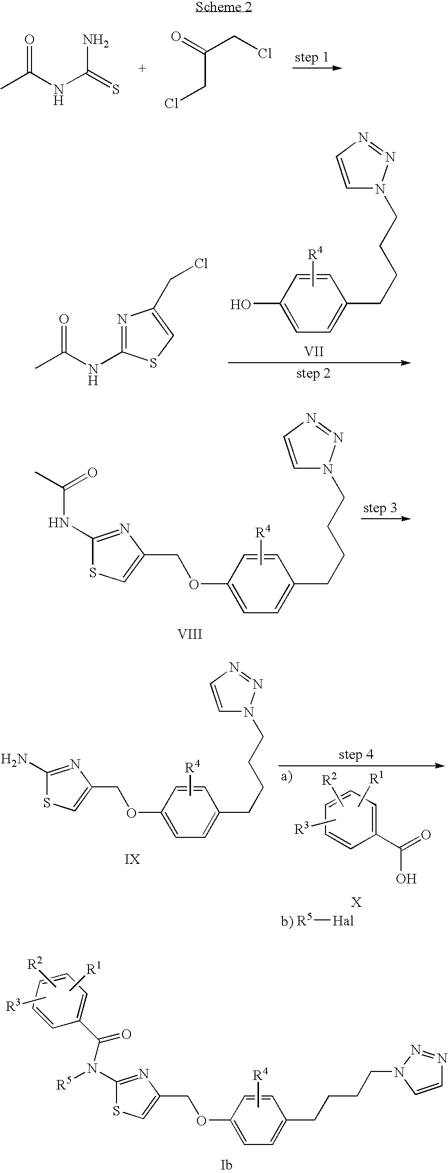

In scheme 2, R¹, R², R³ and R⁴ have the significance given herein before for formula I and R⁵ is hydrogen or alkyl.

Step 1

N-acetylated thiourea and 1,3-dichloroacetone are subjected to a condensation/dehydration sequence yielding the N-acetylated 2-amino-4-chloromethylthiazole. Typical solvents for reactions of this kind are toluene, benzene, acetone and chloroform. If desired the reaction can be carried out under solvent free conditions. The reaction temperatures may vary from 50° C. to 150° C.

Step 2

The thiazole derivatives of formula VIII can be obtained by reactions well known to someone skilled in the art, e.g. by alkylation of 4-(4-[1,2,3]triazol-1-yl)phenol of formula VII with N-acetylated 2-amino-4-chloromethylthiazole. Typically the alkylation is carried out in the presence of potassium iodide or sodium iodide in solvents like methanol, ethanol, isopropanol, acetone, 2-butanone and DMF. Typical bases for this reaction are sodium methylate, sodium hydride, lithium diisopropylamide and cesium carbonate. The reaction temperatures may vary from 50° C. to 150° C. Yields can be improved by use of an excess of the phenol and reisolation of the unreacted reactant.

Step 3

The thiazoles derivatives of formula IX are further obtained by deacetylation either under basic or acidic conditions. Methods of deacetylation are described in the literature and well known to those skilled in the art. Typical bases are NaOH, KOH or LiOH and typical acids are HCl or H₂SO₄. The reactions were carried out in solvents like water, methanol, ethanol or 2-propanol. The reaction temperatures may vary from room temperature to 100° C.

Step 4

The obtained anilines of formula IX are reacted with carboxylic acids of formula X using standard methods for someone skilled in the art, e.g. by activating the carboxylic group in the compounds of formulaX with EDCI, CDI, HOBt or thionylchloride in solvents like THF, dichloromethane, DMF or mixtures thereof and at temperatures varying from −30° C. to 50° C., yielding derivatives of formula Ib wherein R⁵ is hydrogen (part reaction a)).

When the synthesis is further proceeded by reaction b) in step 4 the compounds of formula Ib wherein R⁵ is alkyl are obtained. The alkylation of amides is typically achieved with alkyl halides such as for example the alkyl halides of the formula R⁵-Hal, wherein “Hal” is a halogen-atom, preferably iodine or bromine and R⁵ is alkyl. The reaction is carried out in the presence of a base like NaOH, KOH, triethyl amine or sodium hydride and in solvents like acetone, ethyl acetate, methanol, ethanol, DMF or mixtures thereof at temperatures varying from 0° C. to 150° C.

The compounds of formula I can contain one or several chiral centers and can then be present in a racemic or in an optically active form. The racemates can be separated according to known methods into the enantiomers. For instance, diastereomeric salts which can be separated by crystallization are formed from the racemic mixtures by reaction with an optically active acid such as e.g. D- or L-tartaric acid, mandelic acid, malic acid, lactic acid or camphorsulfonic acid. Alternatively separation of the enantiomers can also be achieved by using chromatography on chiral HPLC-phases which are commercially available.

Pharmacological Activity

The compounds of formula I and their pharmaceutically acceptable salts possess valuable pharmacological properties. It has been found that said compounds inhibit the HER-signaling pathway and show anti-proliferative activity. Consequently the compounds of the present invention are useful in the therapy and/or prevention of illnesses with known over-expression of receptor tyrosine kinases of the HER-family like HER-2 and EGFR (HER-1), especially in the therapy and/or prevention of illnesses mentioned above. The activity of the present compounds as HER-signaling pathway inhibitors is demonstrated by the following biological assay:

Inhibition of HER-2 Phosphorylation in Calu-3 Tumor Cell Line

2×10⁵ Calu-3 (ATTC HTB-55) cells per well were plated in a 12-well plate. After 4 days cells were starved for 16 h in Dulbecco's Modified Eagle Medium (DMEM)/0.5% Fetal Calf Serum (FCS)/1% Glutamine. During this 16 h period cells were incubated with a solution of the test compound in dimethylsulfoxide(DMSO), so that the final concentration of the compound is 1 μM and the final volume of DMSO is 0.5%. Afterwards cells were lysed in lyses buffer containing 1% Triton®X-100, 10% Glycerol, 1 mM Ethylene glycol-bis(2-aminoethylether)-N,N,N′,N′-tetraacetic acid (EGTA), 1.5 mM MgCl₂, 150 mM NaCl, 50 mM 4-(2-hydroxyethyl)-1-piperazineethanesulfonic acid (HEPES) buffer pH 7.5, 1 mM Phenylmethylsulfonyl fluoride (PMSF), 10 ∥g/mL Aprotinin and 0.4 mm Orthovanadate. Cell lysates were analyzed on a Sodium Dodecyl Sulfate Polyacrylamide Gel Electrophoresis (SDS PAGE) and after transfer to a nitrocellulose membrane detected with an antibody specifically recognizing the pY 1248 in HER-2. Inhibition of HER-2 phosphorylation is calculated as percentage of the control, which is treated with DMSO only.

With all compounds a significant inhibition of HER-2-phosphorylation was detected, which is exemplified by the compounds shown in Table 1. The reference compound as used herein is 1-[4-(4-{2-[2-(4-Trifluoromethyl-phenyl)-vinyl]-oxazol-4-ylmethoxy}-phenyl)-butyl]-1H-[1,2,3]triazole (Example 4, p. 88, WO 01/77107).

TABLE 1

| | | Percent inhibition of HER2- |
|---|---|---|
| | Control | phosphorylation |
| | (DMSO) | (compound concentration 1 μM) |
| reference compound | 0 | 52 |
| example 11 | 0 | 69 |
| example 14 | 0 | 72 |
| example 10 | | 75 |
| example 39 | | 87 |
| example 2, 9, 22, 31 | 0 | 55-70 |
| example 36, 37, 40, | 0 | 70-80 |
| 52-5 | | |
| example 13, 41, 43, | 0 | >80 |
| 52-21 | | |

Antiproliferative Activity

The activity of the present compounds as antiproliferative agents is demonstrated by the following biological assay:

Viability Assay of HEK293 Cells

A viability assay was performed using the CellTiter-Glo™ Luminescent Cell Viability Assay (see Promega Corporation's Technical Publication No. 288, pp. 1-11 [revised 2.04] which is hereby incorporated by reference in its entirety). This assay is a homogeneous method of determining the number of viable cells in culture based on quantitation of the ATP present, which signals the presence of metabolically active cells. The assay is designed for use with multiwell formats, making it ideal for automated high-throughput screening (HTS), cell proliferation and cytotoxicity assays. The homogeneous assay procedure involves adding a single reagent (containing luciferase, luciferan substrate, and buffer) directly to cells cultured in serum-supplemented medium. Cell washing, removal of medium and multiple pipetting steps are not required. The system detects as few as 15 cells/well in a 384-well format in 10 minutes after adding reagent and mixing.

The homogeneous “add-mix-measure” format results in cell lysis and generation of a luminescent signal proportional to the amount of ATP present. The amount of ATP is directly proportional to the number of cells present in culture. The above-referenced assay generates a “glow-type” luminescent signal, produced by the luciferase reaction, which has a half-life generally greater than five hours, depending on cell type and medium used. The extended half-life eliminates the need to use reagent injectors and provides flexibility for continuous or batch mode processing of multiple plates. The unique homogeneous format avoids errors that may be introduced by other ATP measurement methods that require multiple steps.

HEK293 cells (human embryonic kidney cell line transformed by Adenovirus 5 fragments, ATCC-No. CRL 1573) were cultivated in Dulbecco's Modified Eagle Medium (DMEM) (1×) liquid (high glucose) (which includes L-Alanyl-L-Glutamine [a stabilized a form of L-Glutamine], 4500 mg/L glucose, and 110 mg/L sodium pyruvate) from Invitrogen Corporation (Invitrogen Catalog Number 31966-021 [now 10569-010] which is hereby incorporated by reference in its entirety), 5% Fetal Calf Serum (FCS, Sigma Cat-No. F4135 (FBS) which is hereby incorporated by reference in its entirety), and 100 Units/ml penicillin/100 μg/ml streptomycin(=Pen/Strep from Invitrogen Cat. No. 15140 which is hereby incorporated by reference in its entirety). For the assay the cells were seeded in 384 well plates, 5000 cells per well, in the same medium. The next day the test compounds were added in various concentrations ranging from 3 μM to 0.00015 μM (10 concentrations, 1:3 diluted). After 7 days the above viability assay was performed in accordance with the following steps:
Step 1: The cell-plate was equilibrated to room temperature for approximately 30 minutes and than the assay reagent was added.
Step 2: The contents were carefully mixed for 15 minutes to induce cell lysis.
Step 3: After 45 minutes the luminescent signal was measured in Victor 2, (scanning multiwell spectrophotometer, Wallac).
   Details:
   1^{st} Day:
Medium: Dulbecco's Modified Eagle Medium (DMEM) (1×) liquid (high glucose) (which includes L-Alanyl-L-Glutamine [a stabilized a form/source of L-Glutamine], 4500 mg/L glucose, and 110 mg/L sodium pyruvate) from Invitrogen Corporation (Invitrogen Catalog Number 31966-021 [now 10569-010]), 5% Fetal Calf Serum (FCS, Sigma Cat-No. F4135 (FBS)), and Pen/Strep (Invitrogen Cat. No. 15140).
HEK293 (ATCC-No. CRL 1573): 5000 cells in 60 μl per well of 384 well plate. (Greiner 781098, white plates)
Incubate 24 h at 37° C., 5% CO₂
   2^{nd} Day: Induction (Substance Testing):

In general the dilution steeps are 1:3

a) Add 8 μl of 10 mM stock solution of compound to 72 μl DMSO

b) dilute 9×1:3 (always 30 μl to 60 μl DMSO) in this DMSO dilution row (results in 10 wells with concentrations from 1000 μM to 0.06 μM)

c) dilute each concentration 1: 4.8 (10 μl compound dilution to 38 μl medium)

d) dilute each concentration 1: 10 (10 μl compound dilution to 90 μmedium)

e) add 10 μl of every concentration to 60 μl medium in the cell plate

resulting in final concentration of DMSO: 0.3% in every well
and resulting in final concentration of compounds from 3 μM to 0.00015 μM
Incubate 168 h (7 days) at 37° C., 5% CO₂
   Analysis:
Add 30 μl of reagent cited above (containing luciferase, luciferan substrate, and buffer),
shake 15 minutes at room temperature
incubate further 45 minutes at room temperature without shaking.
   Measurement:

Victor 2 scanning multiwell spectrophotometer (Wallac), Luminescence mode
Determine IC50 by curve fitting using XLfit® software (ID Business Solution Ltd., Guilford, Surrey, UK) which his hereby incorporated by reference in its entirety.

A significant inhibition of HEK293 cell viability was detected, which is exemplified by the compounds shown in Table 1.

TABLE 1
Results:

| Examples | IC50 HEK293 [nM] |
|---|---|
| 4 | 127 |
| 9 | 116 |
| 24 | 1063 |
| 5, 8, 10, 11, 12, 13, 21, 25, 27, 30, 33, 36, | 5-500 |
| 39, 40, 41, 42, 44, 46, 48, 51, 52-1, 52-4, | |
| 52-5, 52-6, 52-7, 52-9, 52-11, 52-12, 52- | |
| 14, 52-15, 52-19, 52-21, 52-22, 52-23, | |
| 52-25, 52-26 | |
| 6, 14, 16, 20, 28, 29, 31, 43, 52-2, 52-16 | 500-3000 |

The compounds according to this invention and their pharmaceutically acceptable salts can be used as medicaments, e.g. in the form of pharmaceutical compositions. The pharmaceutical compositions can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions.

The above-mentioned pharmaceutical compositions can be obtained by processing the compounds according to this invention with pharmaceutically inert, inorganic or organic carriers. For example, lactose, corn starch or derivatives thereof, talc, stearic acids or it's salts and the like can be used as carriers for tablets, coated tablets, dragées and hard gelatine capsules. Suitable carriers for soft gelatine capsules are, for example, vegetable oils, waxes, fats, semi-solid and liquid polyols and the like. However, depending on the nature of the active substance, carriers may not be required for some soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Preferred pharmaceutical compositions comprise the following:

a) Tablet Formulation (Wet Granulation):

| Item | Ingredients | mg/tablet | | | |
|---|---|---|---|---|---|
| 1. | Compound of formula (I) | 5 | 25 | 100 | 500 |
| 2. | Lactose Anhydrous DTG | 125 | 105 | 30 | 150 |
| | (direct tabletting grade) | | | | |
| 3. | Sta-Rx 1500 (pre- | 6 | 6 | 6 | 30 |
| | gelatinized starch powder) | | | | |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

Manufacturing Procedure:
1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

b) Capsule Formulation:

| Item | Ingredients | mg/capsule | | | |
|---|---|---|---|---|---|
| 1. | Compound of formula (I) | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

Manufacturing Procedure:
1. Mix items 1, 2 and 3 in a suitable mixer for 30 minutes.
2. Add items 4 and 5 and mix for 3 minutes.
3. Fill into a suitable capsule.
c) Micro Suspension
1. Weigh 4.0 g glass beads in custom made tube GL 25, 4 cm (the beads fill half of the tube).
2. Add 50 mg compound, disperse with spatulum and vortex.
3. Add 2 ml gelatin solution (weight beads: gelatin solution=2:1) and vortex.
4. Cap and wrap in aluminium foil for light protection.
5. Prepare a counter balance for the mill.
6. Mill for 4 hours, 20/s in a Retsch mill (for some substances up to 24 hours at 30/s).
7. Extract suspension from beads with two layers of filter (100 μm) on a filter holder, coupled to a recipient vial by centrifugation at 400 g for 2 min.
8. Move extract to measuring cylinder.
9. Repeat washing with small volumes(here 1 ml steps) until final volume is reached or extract is clear.
10. Fill up to final volume with gelatin and homogenize.

The above described preparation yields micro-suspensions of the compounds of formula I-A with particle sizes between 1 and 10 μm. The suspensions are suitable for oral applications and can be used in the in vivo assay described above.

Pharmaceutical compositions containing a compound of the present invention or a pharmaceutically acceptable salt thereof and a therapeutically inert carrier are also an object of the present invention, as is a process for their production, which comprises bringing one or more compounds of the present invention and/or pharmaceutically acceptable salts and, if desired, one or more other therapeutically valuable substances into a galenical administration form together with one or more therapeutically inert carriers.

In accordance with the invention, the compounds of the present invention as well as their pharmaceutically acceptable salts are useful in the control or prevention of illnesses. Based on their HER-signaling pathway inhibition and their antiproliferative activity, said compounds are useful for the treatment of diseases such as cancer in humans or animals and for the production of corresponding pharmaceutical compositions. The dosage depends on various factors such as the manner of administration, species, age and/or individual state of health.

Another embodiment of the invention is a pharmaceutical composition, containing one or more compounds of formula I together with pharmaceutically acceptable excipients.

Still another embodiment of the invention is said pharmaceutical composition for the inhibition of tumor growth.

Still another embodiment of the invention is the use of a compound of formula I for the treatment of cancer.

Still another embodiment of the invention is the use of a compound of formula I for the manufacture of corresponding pharmaceutical compositions for the inhibition of tumor growth.

The following examples and references are provided to aid the understanding of the present invention, the true scope of which is set forth in the appended claims. It is understood that modifications can be made in the procedures set forth without departing from the spirit of the invention.

### EXAMPLE 1

### 2-[4-(4-[1,2,3]Triazol-1-yl-butyl)-phenoxymethyl]-oxazole-4-carboxylic acid benzo [1,3] dioxol-5-yl-amide

100 mg (0.292 mmol) 2-[4-(4-[1,2,3]Triazol-1-yl-butyl)-phenoxymethyl]-oxazole-4-carboxylic acid are dissolved in 2 ml dichloromethane/tetrahydrofuran (1:1) and heated to 45° C. After the addition of 52.1 mg (0.321 mmol) 1,1′ Carbonyldiimidazole the mixture is stirred for 45 min. Then 44.1 mg (0.321 mmol) Benzo[1,3]dioxol-5-ylamine are added and the reaction mixture is stirred for 16 h at room temperature. The reaction mixture is extracted twice with 6 ml saturated sodium hydrogen carbonate solution. The organic layer is evaporated and the residue is purified by preparative HPLC-MS to give the title compound.

Yield: 57 mg (38%).

¹H-NMR (400 MHz, D₆-DMSO): δ=10.13(s, 1H, NH), 8.79(s, 1H, 5-H oxazole), 8.10(s, 1H, triazole), 7.70(s, 1H, triazole), 7.44(d, 1H, 6-H-benzo[1,3]dioxole), 7.27 (dd, 1H, 4-H-benzo[1,3]dioxole), 7.12 (d, 2H, Ar—H, phenoxy), 6.96 (d, 2H, Ar—H, phenoxy), 6.88 (d, 1H, 3-benzo[1,3]dioxole, 5.998 (s, 2H, CH₂-oxol), 5.26 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=462.3(API+)

### EXAMPLE 2

### 2-[4-(4-[1,2,3]Triazol-1-yl-butyl)-phenoxymethyl]-oxazole-4-carboxylic acid (4-chloro-phenyl)-methyl-amide

99.1 mg (0.290 mmol) 2-[4-(4-[1,2,3]Triazol-1-yl-butyl)-phenoxymethyl]-oxazole-4-carboxylic acid are stirred in 10 ml dichloromethane to give a suspension. After the addition of 83.3 mg (0.434 mmol) (3-Dimethylamino-propyl)-ethyl-carbodiimide hydrochloride, 66.5 mg (0.434 mmol) 1-Hydroxy-benzotriazole hydrate and 60.5 μl (0.434 mmol) triethylamine the mixture is stirred for 30 min at room temperature. Then 35.1 μl (0.290 mmol) (4-Chloro-phenyl)-methyl-amine are given to the reaction mixture and stirred for 16 h at room temperature. After addition of 10 ml 1N HCl the organic phase is separated and extracted twice with water. The organic layer is evaporated and the residue is purified by preparative HPLC-MS to give the title compound.

Yield: 55 mg (41%).

¹H-NMR (400 MHz, D₆-DMSO): δ=8.10 (s, 1H, triazole), 8.07 (s, 1H, 5-H oxazole), 7.70(s, 1H, triazole), 7.42-7.38 (m, 2H, Ar—H, 4-Cl-phenyl), 7.30-7.27 (m, 2H, Ar—H, 4-Cl-phenyl), 7.07 (d, 2H, Ar—H, phenoxy), 6.81 (d, 2H, Ar—H, phenoxy), 5.06 (s, 2H, CH₂—O-Ph), 4.39 (t, 2H, 1H-butyl), 3.34 (s, 3H, N—CH₃), 2.54 (t, 2H, 4H-butyl), 1.81 (m, 2H, 2H-butyl), 1.48 (m, 2H, 3H-butyl).

MS: M=466.1 (API+)

### EXAMPLE 3

### 4-[4-(4-[1,2,3]Triazol-1-yl-butyl)-phenoxymethyl]-thiazole-2-carboxylic acid (4-chloro-phenyl)-amide

100 mg (0.292 mmol) 4-[4-(4-[1,2,3]Triazol-1-yl-butyl)-phenoxymethyl]-thiazole-2-carboxylic acid are activated with 42 μl (0.584 mmol) thionyl chloride. The mixture is heated to 45° C. and stirred for 1 hour. After evaporation to dryness the residue is dissolved in 6 ml dichloromethane/tetrahydrofuran (1:1). 37 mg (0.292 mmol) 4-Chloro-phenyl amine are added and the resulting reaction mixture is stirred for 16 h at room temperature. The precipitate is collected and washed twice with ethyl ether to give the title compound.

Yield 90 mg (66%).

¹H-NMR (400 MHz, MeOD): δ=8.42(s, 1H, thiazole), 8.29(s, 1H, triazole), 7.89(s, 1H, triazole), 7.80-7.78 (m, 2H, Ar—H, 4-Cl-phenyl), 7.41-7.38 (m, 2H, Ar—H, 4-Cl-phenyl), 7.14 (d, 2H, Ar—H, phenoxy), 6.97 (d, 2H, Ar—H, phenoxy), 5.26 (s, 2H, CH₂—O-Ph), 4.61 (t, 2H, 1H-butyl), 2.65 (t, 2H, 4H-butyl), 2.01 (m, 2H, 2H-butyl), 1.65 (m, 2H, 3H-butyl).

MS: M=468.0 (API+)

### EXAMPLE 4

### 2-[4-(4-[1,2,3]Triazol-1-yl-butyl)-phenoxymethyl]-oxazole-4-carboxylic acid (2-nitro-4-trifluoromethyl-phenyl)-amide

The title compound is prepared from 30 mg (0.146 mmol) 2-[4-(4-[1,2,3]Triazol-1-yl-butyl)-phenoxymethyl]-oxazole-4-carboxylic acid and 52.3 mg (0.153 mmol) 2-Nitro-4-trifluoromethyl-phenylamine as described in Example 1. Yield 24 mg (31%).

¹H-NMR (400 MHz, D₆-DMSO): δ=11.45 (s, 1H), 9.025(d, 1H), 8.64-8.59(m, 1H), 8.45(s, 1H,), 8.19(m, 1H), 8.10(s, 1H), 7.70(s, 1H), 7.13 (d, 2H,), 6.99 (d, 2H), 5.32 (s, 2H), 4.39 (t, 2H), 2.54 (t, 2H), 1.81 (m, 2H,), 1.48 (m, 2H).

MS: M=531.3 (API+)

### EXAMPLE 5

### 2-[4-(4-[1,2,3]Triazol-1-yl-butyl)-phenoxymethyl]-oxazole-4-carboxylic acid (3,5-difluoro-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 29 mg (0.225 mmol) 3,5-Difluoro-phenylamine as described in Example 1. Yield 17 mg (18%).

¹H-NMR (400 MHz, MeOD): δ=8.58(s, 1H, oxazole), 7.96(s, 1H, triazole), 7.72(s, 1H, triazole), 7.47 (m, 2H, 2,6-H-3,5-F-Ph), 7.13 (d, 2H, Ar—H, phenoxy), 6.96 (d, 2H, Ar—H, phenoxy), 6.73 (m, 1H, 4-H-3,5-F-Ph), 5.24 (s, 2H, CH₂—O-Ph), 4.46 (t, 2H, 1H-butyl), 2.62 (t, 2H, 4H-butyl), 1.93 (m, 2H, 2H-butyl), 1.59 (m, 2H, 3H-butyl).

MS: M=454.3 (API+)

### EXAMPLE 6

### 2-[4-(4-[1,2,3]Triazol-1-yl-butyl)-phenoxymethyl]-oxazole-4-carboxylic acid (4-chloro-phenyl)-amide

The title compound is prepared from 100 mg (0.292 mmol) 2-[4-(4-[1,2,3]Triazol-1-yl-butyl)-phenoxymethyl]-oxazole-4-carboxylic acid and 41 mg (0.321 mmol) 4-Chloro-phenylamine as described in Example 1. Yield 57 mg (39%).

¹H-NMR (400 MHz, D₆-DMSO): δ=10.39 (s, 1H, NH), 8.84 (s, 1H, oxazole), 8.10 (s, 1H, triazole), 7.86 (d, 2H, Ar—H, 4-Cl-phenyl), 7.70 (s, 1H, triazole), 7.40 (d, 2H, Ar—H, 4-Cl-phenyl), 7.12 (d, 2H, Ar—H, phenoxy), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=452.3 (API+)

### EXAMPLE 7

### 2-[4-(4-[1,2,3]Triazol-1-yl-butyl)-phenoxymethyl]-oxazole-4-carboxylic acid (2,4-difluoro-phenyl)-amide

The title compound is prepared from 100 mg (0.292 mmol) 2-[4-(4-[1,2,3]Triazol-1-yl-butyl)-phenoxymethyl]-oxazole-4-carboxylic acid and 41 mg (0.321 mmol) 2,4-Difluoro-phenylamine as described in Example 1. Yield 34 mg (23%).

MS: M 454.2 (API+)

### EXAMPLE 8

### 2-[4-(4-[1,2,3]Triazol-1-yl-butyl)-phenoxymethyl]-oxazole-4-carboxylic acid (4-difluoromethylsulfanyl-phenyl)-amide

The title compound is prepared from 100 mg (0.292 mmol) 2-[4-(4-[1,2,3]Triazol-1-yl-butyl)-phenoxymethyl]-oxazole-4-carboxylic acid and 56 mg (0.321 mmol) 4-Difluoromethylsulfanyl-phenylamine as described in Example 1. Yield 45 mg (28%).

¹H-NMR (400 MHz, D₆-DMSO): δ=10.47 (s, 1H, NH), 8.86 (s, 1H, oxazole), 8.10 (s, 1H, triazole), 7.92 (m, 2H, Ar—H, 4-F₂HC-thiophenyl), 7.70 (s, 1H, triazole), 7.56 (m, 2H, Ar—H, 4-F₂HC-thiophenyl), 7.55-7.28 (m, 1H, F₂HC), 7.12 (d, 2H, Ar—H, phenoxy), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=500.3 (API+)

### EXAMPLE 9

### 2-[4-(4-[1,2,3]Triazol-1-yl-butyl)-phenoxymethyl]-oxazole-4-carboxylic acid (2,2-difluoro-benzo [1,3] dioxol-5-yl)-amide

The title compound is prepared from 100 mg (0.292 mmol) 2-[4-(4-[1,2,3]Triazol-1-yl-butyl)-phenoxymethyl]-oxazole-4-carboxylic acid and 55 mg (0.321 mmol) 5-Amino-2,2-difluoro-1,3-benzodioxole as described in Example 1. Yield 64 mg (40%).

¹H-NMR (400 MHz, D₆-DMSO): δ=10.49 (s, 1H, NH), 8.85 (s, 1H, oxazole), 8.11 (s, 1H, triazole), 7.92-7.92 (m, 1H, Ar—H, benzo[1,3]dioxole), 7.71 (s, 1H, triazole), 7.40-7.37 (m, 1H, Ar—H, benzo[1,3]dioxole), 7.33-7.30 (m, 1H, Ar—H, benzo[1,3]dioxole), 7.55-7.28 (m, 1H, F₂HC), 7.12 (d, 2H, Ar—H, phenoxy), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=498.3 (API+)

### EXAMPLE 10

### 2-[4-(4-[1,2,3]Triazol-1-yl-butyl)-phenoxymethyl]-oxazole-4-carboxylic acid (4-trifluoro-methoxy-phenyl)-amide

The title compound is prepared from 100 mg (0.292 mmol) 2-[4-(4-[1,2,3]Triazol-1-yl-butyl)-phenoxymethyl]-oxazole-4-carboxylic acid and 57 mg (0.321 mmol) 4-Trifluoromethoxy-phenylamine as described in Example 1. Yield 60 mg (37%).

¹H-NMR (400 MHz, D₆-DMSO): δ=10.46 (s, 1H, NH), 8.85 (s, 1H, oxazole), 8.10 (s, 1H, triazole), 7.93 (d, 2H, Ar—H, 4-F₃C—O-phenyl), 7.70 (s, 1H, triazole), 7.36 (dm, 2H, Ar—H, 4-F₃C—O-phenyl), 7.12 (d, 2H, Ar—H, phenoxy), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=502.3 (API+)

### EXAMPLE 11

### 2-[4-(4-[1,2,3]Triazol-1-yl-butyl)-phenoxymethyl]-oxazole-4-carboxylic acid (4-trifluoro-methylsulfanyl-phenyl)-amide

The title compound is prepared from 100 mg (0.292 mmol) 2-[4-(4-[1,2,3]Triazol-1-yl-butyl)-phenoxymethyl]-oxazole-4-carboxylic acid and 62 mg (0.321 mmol) 4-Trifluoromethylsulfanyl-phenylamine as described in Example 1. Yield 8 mg (5%).

¹H-NMR (400 MHz, D₆-DMSO): δ=10.57 (s, 1H, NH), 8.88 (s, 1H, oxazole), 8.10 (s, 1H, triazole), 7.99 (m, 2H, Ar—H, 4-F₃C—S-phenyl), 7.71-7.69 (m, 2H, Ar—H, 4-F₃C—S-phenyl), 7.70 (s, 1H, triazole), 7.12 (d, 2H, Ar—H, phenoxy), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=518.2 (API+)

### EXAMPLE 12

### 2-[4-(4-[1,2,3]Triazol-1-yl-butyl)-phenoxymethyl]-oxazole-4-carboxylic acid (3-chloro-4-fluoro-phenyl)-amide

The title compound is prepared from 100 mg (0.292 mmol) 2-[4-(4-[1,2,3]Triazol-1-yl-butyl)-phenoxymethyl]-oxazole-4-carboxylic acid and 62 mg (0.321 mmol) 3-Chloro-4-fluoro-phenylamine as described in Example 1. Yield 15 mg (10%).

¹H-NMR (400 MHz, D₆-DMSO): δ=10.50 (s, 1H, NH), 8.85 (s, 1H, oxazole), 8.12 (m, 1H, 2-H-3-Cl-4-F-phenyl), 8.11 (s, 1H, triazole), 7.80 (m, 1H, 6-H-3-Cl-4-F-phenyl), 7.70 (s, 1H, triazole), 7.12 (d, 2H, Ar—H, phenoxy), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=470.2 (API+)

### EXAMPLE 13

### 2-[4-(4-[1,2,3]Triazol-1-yl-butyl)-phenoxymethyl]-oxazole-4-carboxylic acid (4-trifluoromethyl-phenyl)-amide

The title compound is prepared from 100 mg (0.292 mmol) 2-[4-(4-[1,2,3]Triazol-1-yl-butyl)-phenoxymethyl]-oxazole-4-carboxylic acid and 52 mg (0.321 mmol) 4-trifluoromethyl-phenylamine as described in Example 1. Yield 102 mg (65%).

¹H-NMR (400 MHz, D₆-DMSO): δ=10.61 (s, 1H, NH), 8.88 (s, 1H, oxazole), 8.11 (s, 1H, triazole), 8.05 (m, 2H, Ar—H, 4-CF₃-phenyl), 7.71 (m, 2H, Ar—H, 4-CF₃-phenyl), 7.70 (s, 1H, triazole), 7.12 (d, 2H, Ar—H, phenoxy), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=486.0 (API+)

### EXAMPLE 14

### 2-[4-(4-[1,2,3]Triazol-1-yl-butyl)-phenoxymethyl]-oxazole-4-carboxylic acid (4-chloro-2-fluoro-phenyl)-amide

The title compound is prepared from 100 mg (0.292 mmol) 2-[4-(4-[1,2,3]Triazol-1-yl-butyl)-phenoxymethyl]-oxazole-4-carboxylic acid and 47 mg (0.321 mmol) 3-Chloro-4-fluoro-phenylamine as described in Example 1. Yield 15 mg (10%).

¹H-NMR (400 MHz, D₆-DMSO): δ=9.95 (s, 1H, NH), 8.87 (s, 1H, oxazole), 8.10 (s, 1H, triazole), 7.76-7.72 (m, 1H, 6-H-4-Cl-2-F-phenyl), 7.70 (s, 1H, triazole), 7.56-7.53 (m, 1H, 3-H-4-Cl-2-F-phenyl), 7.33-7.31 (m, 1H, 5-H-4-Cl-2-F-phenyl), 7.12 (d, 2H, Ar—H, phenoxy), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=470.3 (API+)

### EXAMPLE 15

### 2-[4-(4-[1,2,3]Triazol-1-yl-butyl)-phenoxymethyl]-oxazole-4-carboxylic acid (4-(thiazol-2-ylsulfamoyl)-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 52 mg (0.204 mmol) 4-Amino-N-thiazol-2-yl-benzenesulfonamide as described in Example 1. Yield 7 mg (6%).

¹H-NMR (400 MHz, D₆-DMSO): δ=10.54 (s, 1H, NH), 8.87 (s, 1H, oxazole), 8.10 (s, 1H, triazole), 7.97-7.94 (m, 2H, Ar—H, SO₂-phenyl), 7.77-7.75. (m, 2H, Ar—H, SO₂-phenyl), 7.70 (s, 1H, triazole), 7.24 (d, 1H, thiazole), 7.12 (d, 2H, Ar—H, phenoxy), 6.97 (d, 2H, Ar—H, phenoxy), 6.81 (d, 1H, thiazole), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=580.4 (API+)

### EXAMPLE 16

### 2-[4-(4-[1,2,3]Triazol-1-yl-butyl)-phenoxymethyl]-oxazole-4-carboxylic acid [4-(5-methyl-thiadiazol-2-ylsulfamoyl)-phenyl]-amide

The title compound is prepared from 70 mg (0.204 mmol) 2-[4-(4-[1,2,3]Triazol-1-yl-butyl)-phenoxymethyl]-oxazole-4-carboxylic acid and 55 mg (0.204 mmol) 4-Amino-N-(5-methyl-[1,3,4]thiadiazol-2-yl)-benzenesulfonamide as described in Example 1. Yield 10 mg (8%).

MS: M=595.3 (API+)

### EXAMPLE 17

### 2-[4-(4-[1,2,3]Triazol-1-yl-butyl)-phenoxymethyl]-oxazole-4-carboxylic acid [4-(5-methyl-isoxazol-3-ylsulfamoyl)-phenyl]-amide

The title compound is prepared from 70 mg (0.204 mmol) 2-[4-(4-[1,2,3]Triazol-1-yl-butyl)-phenoxymethyl]-oxazole-4-carboxylic acid and 52 mg (0.204 mmol) 4-Amino-N-(5-methyl-isoxazol-3-yl)-benzenesulfonamide as described in Example 1. Yield 11 mg (9%).

MS: M=578.4 (API+)

### EXAMPLE 18

### 2-[4-(4-[1,2,3]Triazol-1-yl-butyl)-phenoxymethyl]-oxazole-4-carboxylic acid (4-acetylsulfamoyl-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 44 mg (0.204 mmol) N-Acetyl-4-amino-benzenesulfonamide as described in Example 1. Yield 9 mg (8%).

MS: M=539.2 (API+)

### EXAMPLE 19

### 2-[4-(4-[1,2,3]Triazol-1-yl-butyl)-phenoxymethyl]-oxazole-4-carboxylic acid (4-bromo-2-fluoro-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 39 mg (0.204 mmol) 4-Bromo-2-fluoro-phenylamine as described in Example 1. Yield 65 mg (62%).

¹H-NMR (400 MHz, D₆-DMSO): δ=9.93 (s, 1H, NH), 8.87 (s, 1H, oxazole), 8.11 (s, 1H, triazole), 7.71-7.69 (m, 1H, 6-H-2-F-4-Br-phenyl), 7.70 (s, 1H, triazole), 7.67-7.64 (m, 1H, 3-H-2-F-4-Br-phenyl), 7.24 (d, 1H, thiazole), 7.457-7.43 (m, 1H, 5-H-2-F-4-Br-phenyl), 7.12 (d, 2H, Ar—H, phenoxy), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=516.2 (API+)

### EXAMPLE 20

### 2-[4-(4-[1,2,3]Triazol-1-yl-butyl)-phenoxymethyl]-oxazole-4-carboxylic acid (3-fluoro-2-methyl-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 26 mg (0.204 mmol) 3-fluoro-2-methyl-phenylamine as described in Example 1. Yield 35 mg (38%).

¹H-NMR (400 MHz, D₆-DMSO): δ=9.92 (s, 1H, NH), 8.82 (s, 1H, oxazole), 8.11 (s, 1H, triazole), 7.70 (s, 1H, triazole), 7.30 (d, 1H, 6-H-3-F-2-Br-methyl), 7.24 (dd, 1H, 5-H-3-F-2-Br-methyl), 7.12 (d, 2H, Ar—H, phenoxy), 7.09-7.05 (m, 1H, 4-H-3-F-2-Br-methyl), 6.97 (d, 2H, Ar-H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=450.3 (API+)

### EXAMPLE 21

### 2-[4-(4-[1,2,3]Triazol-1-yl-butyl)-phenoxymethyl]-oxazole-4-carboxylic acid (4-fluoro-3-nitro-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 32 mg (0.204 mmol) 4-fluoro-3-nitro-phenylamine as described in Example 1. Yield 24 mg (24%).

MS: M=481.3 (API+)

### EXAMPLE 22

### 2-[4-(4-[1,2,3]Triazol-1-yl-butyl)-phenoxymethyl]-oxazole-4-carboxylic acid (4-difluoromethoxy-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 32 mg (0.204 mmol) 4-difluoromethoxy-phenylamine as described in Example 1. Yield 41 mg (41%).

¹H-NMR (400 MHz, D₆-DMSO): δ=10.34 (s, 1H, NH), 8.83 (s, 1H, oxazole), 8.10 (s, 1H, triazole), 7.85-7.83 (m, 2H, Ar—H, 4-F₂HCO-phenyl), 7.70 (s, 1H, triazole), 7.17-7.15 (m, 2H, Ar-H, 4-F₂HCO-phenyl), 7.17 (t, 1H, H—CF₂), 7.12 (d, 2H, Ar—H, phenoxy), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=450.3 (API+)

### EXAMPLE 23

### 2-[4-(4-[1,2,3]Triazol-1-yl-butyl)-phenoxymethyl]-oxazole-4-carboxylic acid (3-trifluoromethyl-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 33 mg (0.204 mmol) 3-trifluoromethyl-phenylamine as described in Example 1. Yield 21 mg (21%).

MS: M=486.2 (API+)

### EXAMPLE 24

### 2-[4-(4-[1,2,3]Triazol-1-yl-butyl)-phenoxymethyl]-oxazole-4-carboxylic acid (4-methylsulfanyl-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 28 mg (0.204 mmol) 4-methylsulfanyl-phenylamine as described in Example 1. Yield 22 mg (23%).

MS: M=464.2 (API+)

### EXAMPLE 25

### 2-[4-(4-[1,2,3]Triazol-1-yl-butyl)-phenoxymethyl]-oxazole-4-carboxylic acid (4-fluoro-2-methyl-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 26 mg (0.204 mmol) 4-fluoro-2-methyl-phenylamine as described in Example 1. Yield 10 mg (11%).

¹H-NMR (400 MHz, D₆-DMSO): δ=9.77 (s, 1H, NH), 8.80 (s, 1H, oxazole), 8.10 (s, 1H, triazole), 7.70 (s, 1H, triazole), 7.42-7.38 (m, 1H, 6H-4-F-2-methyl-phenyl), 7.15-7.11 (m, 1H, 3H-4-F-2-methyl-phenyl), 7.12 (d, 2H, Ar—H, phenoxy), 7.06-7.01 (m, 1H, 5H-4-F-2-methyl-phenyl), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=450.3 (API+)

### EXAMPLE 26

### 2-[4-(4-[1,2,3]Triazol-1-yl-butyl)-phenoxymethyl]-oxazole-4-carboxylic acid (4-tert-butyl-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 31 mg (0.204 mmol) 4-tert-butyl-phenylamine as described in Example 1. Yield 16 mg (17%).

¹H-NMR (400 MHz, D₆-DMSO): δ=10.12 (s, 1H, NH), 8.80 (s, 1H, oxazole), 8.10 (s, 1H, triazole), 7.71-7.68 (m, 2H, Ar—H, tBu-phenyl), 7.70 (s, 1H, triazole), 7.36-7.34 (m, 2H, Ar—H, tBu-phenyl), 7.12 (d, 2H, Ar—H, phenoxy), 6.97 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl), 1.27 (s, 9H, tBu).

MS: M=474.4 (API+)

### EXAMPLE 27

### 2-[4-(4-[1,2,3]Triazol-1-yl-butyl)-phenoxymethyl]-oxazole-4-carboxylic acid (4-chloro-3-trifluoromethyl-phenyl)-amide

The title compound is prepared from 70 mg (0.204 mmol) 2-[4-(4-[1,2,3]Triazol-1-yl-butyl)-phenoxymethyl]-oxazole-4-carboxylic acid and 40 mg (0.204 mmol) 4-chloro-3-trifluoromethyl-phenylamine as described in Example 1. Yield 16 mg (15%).

MS: M=520.2 (API+)

### EXAMPLE 28

### 2-[4-(4-[1,2,3]Triazol-1-yl-butyl)-phenoxymethyl]-oxazole-4-carboxylic acid p-tolylamide

The title compound is prepared from 70 mg (0.204 mmol) 2-[4-(4-[1,2,3]Triazol-1-yl-butyl)-phenoxymethyl]-oxazole-4-carboxylic acid and 22 mg (0.204 mmol) p-Tolylamine as described in Example 1. Yield 13 mg (15%).

¹H-NMR (400 MHz, D₆-DMSO): δ=10.10 (s, 1H, NH), 8.80 (s, 1H, oxazole), 8.10 (s, 1H, triazole), 7.70 (s, 1H, triazole), 7.67 (d, 2H, Ar—H, 4-methyl-phenyl), 7.14 (d, 2H, Ar—H, 4-methyl-phenyl), 7.12 (d, 2H, Ar—H, phenoxy), 6.97 (d, 2H, Ar—H, phenoxy), 5.28 (s, 2H, CH₂—O—Ph), 4.39 (t, 2H, 1H-butyl), 2.54 (t, 2H, 4H-butyl), 2.27 (s, 3H, methyl), 1.81 (m, 2H, 2H-butyl), 1.48 (m, 2H, 3H-butyl).

MS: M=432.3 (API+)

### EXAMPLE 29

### 2-[4-(4-[1,2,3]Triazol-1-yl-butyl)-phenoxymethyl]-oxazole-4-carboxylic acid methyl-phenyl-amide

The title compound is prepared from 100 mg (0.292 mmol) 2-[4-(4-[1,2,3]Triazol-1-yl-butyl)-phenoxymethyl]-oxazole-4-carboxylic acid and 31.6 μl (0.292 mmol) Methyl-phenyl-amine as described in Example 2. Yield 87 mg (69%).

MS: M=432.3 (API+)

### EXAMPLE 30

### 2-[4-(4-[1,2,3]Triazol-1-yl-butyl)-phenoxymethyl]-oxazole-4-carboxylic acid methyl-(4-trifluoromethoxy-phenyl)-amide

The title compound is prepared from 100 mg (0.292 mmol) 2-[4-(4-[1,2,3]Triazol-1-yl-butyl)-phenoxymethyl]-oxazole-4-carboxylic acid and 56 mg (0.292 mmol) Methyl-(4-trifluoromethoxy-phenyl)-amine as described in Example 2. Yield 42 mg (28%).

MS: M=516.3 (API+)

### EXAMPLE 31

### 2-[4-(4-[1,2,3]Triazol-1-yl-butyl)-phenoxymethyl]-oxazole-4-carboxylic acid (4-fluoro-phenyl)-amide

The title compound is prepared from 70 mg (0.205 mmol) 2-[4-(4-[1,2,3]Triazol-1-yl-butyl)-phenoxymethyl]-oxazole-4-carboxylic acid and 23 mg (0.205 mmol) 4-Fluoro-phenylamine as described in Example 1. Yield 25 mg (28%).

MS: M=436.2 (API+)

### EXAMPLE 32

### 2-[4-(4-[1,2,3]Triazol-1-yl-butyl)-phenoxymethyl]-oxazole-4-carboxylic acid (4-fluoro-3-methyl-phenyl)-amide

The title compound is prepared from 70 mg (0.205 mmol) 2-[4-(4-[1,2,3]Triazol-1-yl-butyl)-phenoxymethyl]-oxazole-4-carboxylic acid and 26 mg (0.205 mmol) 4-Fluoro-3-methyl-phenylamine as described in Example 1. Yield 2.3 mg (2.5%).

MS: M=450.2 (API+)

### EXAMPLE 33

### 2-[4-(4-[1,2,3]Triazol-1-yl-butyl)-phenoxymethyl]-oxazole-4-carboxylic acid (2-fluoro-phenyl)-amide

The title compound is prepared from 70 mg (0.205 mmol) 2-[4-(4-[1,2,3]Triazol-1-yl-butyl)-phenoxymethyl]-oxazole-4-carboxylic acid and 23 mg (0.205 mmol) 2-Fluoro-phenylamine as described in Example 1. Yield 5 mg (6%).

MS: M=458.4 (API+Na)

### EXAMPLE 34

### 2-[4-(4-[1,2,3]Triazol-1-yl-butyl)-phenoxymethyl]-oxazole-4-carboxylic acid (4-nitro-2-trifluoromethyl-phenyl)-amide

The title compound is prepared from 70 mg (0.205 mmol) 2-[4-(4-[1,2,3]Triazol-1-yl-butyl)-phenoxymethyl]-oxazole-4-carboxylic acid and 42 mg (0.205 mmol) 4-Nitro-2-trifluoromethyl-phenylamine as described in Example 1. Yield 2 mg (1.8%).

MS: M=531.1 (API+)

### EXAMPLE 35

### 2-[4-(4-[1,2,3]Triazol-1-yl-butyl)-phenoxymethyl]-oxazole-4-carboxylic acid (3-fluoro-phenyl)-amide

The title compound is prepared from 70 mg (0.205 mmol) 2-[4-(4-[1,2,3]Triazol-1-yl-butyl)-phenoxymethyl]-oxazole-4-carboxylic acid and 23 mg (0.205 mmol) 3-fluoro-phenylamine as described in Example 1. Yield 11 mg (12%).

¹H-NMR (400 MHz, MeOD): δ=8.44(s, 1H, oxazole), 7.84(s, 1H, triazole), 7.60(s, 1H, triazole), 7.59-7.55 (m, 1H, 2-H-3-F-Ph), 7.28-7.22 (m, 1H, 5-H-3-F-Ph), 7.20-7.14 (m, 1H, 6-H-3-F-Ph), 7.17 (d, 2H, Ar—H, phenoxy), 6.85 (d, 2H, Ar—H, phenoxy), 6.81-6.76 (m, 1H, 4-H-3-F-Ph), 5.13 (s, 2H, CH₂—O-Ph), 4.34 (t, 2H, 1H-butyl), 2.50 (t, 2H, 4H-butyl), 1.81 (m, 2H, 2H-butyl), 1.48 (m, 2H, 3H-butyl).

MS: M=436.2 (API+)

### EXAMPLE 36

### 2-[4-(4-[1,2,3]Triazol-1-yl-butyl)-phenoxymethyl]-oxazole-4-carboxylic acid (3-fluoro-4-methyl-phenyl)-amide

The title compound is prepared from 70 mg (0.205 mmol) 2-[4-(4-[1,2,3]Triazol-1-yl-butyl)-phenoxymethyl]-oxazole-4-carboxylic acid and 26 mg (0.205 mmol) 3-fluoro-4-methyl-phenylamine as described in Example 1. Yield 1.3 mg (1.4%).

MS: M=450.3 (API+)

### EXAMPLE 37

### 2-[4-(4-[1,2,3]Triazol-1-yl-butyl)-phenoxymethyl]-oxazole-4-carboxylic acid (4-methoxy-phenyl)-amide

The title compound is prepared from 70 mg (0.205 mmol) 2-[4-(4-[1,2,3]Triazol-1-yl-butyl)-phenoxymethyl]-oxazole-4-carboxylic acid and 25 mg (0.205 mmol) 3-fluoro-4-methyl-phenylamine as described in Example 1. Yield 37 mg (40%).

¹H-NMR (400 MHz, MeOD): δ=8.39(s, 1H, oxazole), 7.83(s, 1H, triazole), 7.60(s, 1H, triazole), 7.50-7.46 (m, 2H, Ar—H, 4-MeO-Ph), 7.01 (d, 2H, Ar—H, phenoxy), 6.856 (d, 2H, Ar—H, phenoxy), 6.84-6.81 (m, 2H, Ar—H, 4-MeO-Ph), 5.12 (s, 2H, CH₂—O-Ph), 4.34 (t, 2H, 1H-butyl), 2.50 (t, 2H, 4H-butyl), 1.81 (m, 2H, 2H-butyl), 1.48 (m, 2H, 3H-butyl).

MS: M=448.3 (API+)

### EXAMPLE 38

### 2-[4-(4-[1,2,3]Triazol-1-yl-butyl)-phenoxymethyl]-oxazole-4-carboxylic acid (3,4-difluoro-phenyl)-amide

The title compound is prepared from 70 mg (0.205 mmol) 2-[4-(4-[1,2,3]Triazol-1-yl-butyl)-phenoxymethyl]-oxazole-4-carboxylic acid and 26 mg (0.205 mmol) 3,4-Difluoro-phenylamine as described in Example 1. Yield 15 mg (16%).

¹H-NMR (400 MHz, MeOD): δ=8.55(s, 1H, oxazole), 7.96(s, 1H, triazole), 7.88-7.82 (m, 1H, 2-H-3,4-F-phenyl), 7.72(s, 1H, triazole), 7.47-7.44 (m, 1H, 6-H-3,4-F-phenyl), 7.30-7.23 (m, 1H, 5-H-3,4-F-phenyl), 7.13 (d, 2H, Ar—H, phenoxy), 6.97 (d, 2H, Ar—H, phenoxy), 5.24 (s, 2H, CH₂—O-Ph), 4.46 (t, 2H, 1H-butyl), 2.61 (t, 2H, 4H-butyl), 1.93 (m, 2H, 2H-butyl), 1.60 (m, 2H, 3H-butyl).

MS: M=454.0 (API+)

### EXAMPLE 39

### 4-[4-(4-[1,2,3]Triazol-1-yl-butyl)-phenoxymethyl]-thiazole-2-carboxylic acid (4-trifluoromethoxy-phenyl)-amide

The title compound is prepared from 70 mg (0.205 mmol) 4-[4-(4-[1,2,3]Triazol-1-yl-butyl)-phenoxymethyl]-thiazole-2-carboxylic acid and 36 mg (0.205 mmol) 4-Trifluoromethoxy-phenylamine as described in Example 1. Yield 12 mg (11%).

¹H-NMR (400 MHz, MeOD): δ=7.84(s, 1H, thiazole), 7.78(s, 1H, triazole), 7.79-7.76 (m, 2H, 2,6-H-4-CF₃O-phenyl), 7.60(s, 1H, triazole), 7.21-7.18 (m, 2H, 2,6-H-4-CF₃O-phenyl), 7.01 (d, 2H, Ar—H, phenoxy), 6.85 (d, 2H, Ar—H, phenoxy), 5.15 (s, 2H, CH₂—O-Ph), 4.34 (t, 2H, 1H-butyl), 2.50 (t, 2H, 4H-butyl), 1.82 (m, 2H, 2H-butyl), 1.48 (m, 2H, 3H-butyl).

MS: M=518.1 (API+)

### EXAMPLE 40

### 4-[4-(4-[1,2,3]Triazol-1-yl-butyl)-phenoxymethyl]-thiazole-2-carboxylic acid (4-pentafluorosulfanyl-phenyl)-amide

The title compound is prepared from 70 mg (0.205 mmol) 4-[4-(4-[1,2,3]Triazol-1-yl-butyl)-phenoxymethyl]-thiazole-2-carboxylic acid and 45 mg (0.205 mmol) 4-pentafluorosulfanyl-phenylamine as described in Example 1. Yield 2 mg (2%).

MS: M=560.2 (API+)

### EXAMPLE 41

### 2-[4-(4-[1,2,3]Triazol-1-yl-butyl)-phenoxymethyl]-thiazole-4-carboxylic acid (4-trifluoromethyl-phenyl)-amide

The title compound is prepared from 100 mg (0.279 mmol) 2-[4-(4-[1,2,3]Triazol-1-yl-butyl)-phenoxymethyl]-thiazole-4-carboxylic acid and 45 mg (0.279 mmol) 4-Trifluoromethyl-phenylamine as described in Example 3. After stirring 16 h at room temperature, 10 ml 1N HCl are added to the reaction mixture. The organic layer is extracted twice with dichloromethane. The extract is evaporated to give 76 mg (54%) of product.

¹H-NMR (400 MHz, MeOD): δ=8.40(s, 1H, thiazole), 7.99(d, 2H, Ar—H, F₃C-phenyl), 7.76(s, 1H, triazole), 7.72(s, 1H, triazole), 7.69 (d, 2H, Ar—H, F₃C-phenyl), 7.15 (d, 2H, Ar—H, phenoxy), 6.99 (d, 2H, Ar—H, phenoxy), 5.46 (s, 2H, CH₂—O-Ph), 4.46 (t, 2H, 1H-butyl), 2.63 (t, 2H, 4H-butyl), 1.93 (m, 2H, 2H-butyl), 1.61 (m, 2H, 3H-butyl).

MS: M=502.1 (API+)

### EXAMPLE 42

### 2-[4-(4-[1,2,3]Triazol-1-yl-butyl)-phenoxymethyl]-thiazole-4-carboxylic acid (4-chloro-phenyl)-amide

The title compound is prepared from 100 mg (0.279 mmol) 2-[4-(4-[1,2,3]Triazol-1-yl-butyl)-phenoxymethyl]-thiazole-4-carboxylic acid and 36 mg (0.279 mmol) 4-Chloro-phenylamine as described in Example 3. After stirring 16 h at room temperature, 10 ml 1N HCl are added to the reaction mixture. The organic layer is extracted twice with dichloromethane. The extract is evaporated to give 85 mg (65%) of product.

¹H-NMR (400 MHz, D₆-DMSO): δ=10.47 (s, 1H, NH), 8.49(s, 1H, thiazole), 8.11(s, 1H, triazole), 7.89(m, 2H, Ar—H, 4-Cl-phenyl), 7.71(s, 1H, triazole), 7.41 (m, 2H, Ar—H, 4-Cl-phenyl), 7.14 (d, 2H, Ar—H, phenoxy), 7.00 (d, 2H, Ar—H, phenoxy), 5.48 (s, 2H, CH₂—O-Ph), 4.39 (t, 2H, 1H-butyl), 2.55 (t, 2H, 4H-butyl), 1.81 (m, 2H, 2H-butyl), 1.49 (m, 2H, 3H-butyl).

MS: M=468.1 (API+)

### EXAMPLE 43

### 2-[4-(4-[1,2,3]Triazol-1-yl-butyl)-phenoxymethyl]-thiazole-4-carboxylic acid (4-trifluoromethoxy-phenyl)-amide

The title compound is prepared from 100 mg (0.279 mmol) 2-[4-(4-[1,2,3]Triazol-1-yl-butyl)-phenoxymethyl]-thiazole-4-carboxylic acid and 49 mg (0.279 mmol) 4-Trifluoro-methoxy-phenylamine as described in Example 3. After stirring 16 h at room temperature, 10 ml 1N HCl are added to the reaction mixture. The organic layer is extracted twice with dichloromethane. The extract is evaporated to give 98 mg (68%) of product.

¹H-NMR (400 MHz, MeOD): δ=8.40(s, 1H, thiazole), 7.99(d, 2H, Ar—H, F₃CO-phenyl), 7.96(s, 1H, triazole), 7.72(s, 1H, triazole), 7.69 (d, 2H, Ar—H, F₃CO-phenyl), 7.15 (d, 2H, Ar—H, phenoxy), 6.99 (d, 2H, Ar—H, phenoxy), 5.46 (s, 2H, CH₂—O-Ph), 4.46 (t, 2H, 1H-butyl), 2.63 (t, 2H, 4H-butyl), 1.93 (m, 2H, 2H-butyl), 1.61 (m, 2H, 3H-butyl).

MS: M=518.1 (API+)

### EXAMPLE 44

### 2-[4-(4-[1,2,3]Triazol-1-yl-butyl)-phenoxymethyl]-oxazole-4-carboxylic acid (2,6-dichloro-phenyl)-amide

100 mg (0.292 mmol) 2-[4-(4-[1,2,3]Triazol-1-yl-butyl)-phenoxymethyl]-oxazole-4-carboxylic acid and 106 μl (1.460 mmol) thionyl chloride are stirred at 45° C. until development of gas stops. The reaction mixture is evaporated to dryness. The residue is dissolved in 6 ml dichloromethane. After the addition of 47 mg (0.292 mmol) 2,6-Dichloro-phenylamine the reaction mixture is stirred for 16 h at room temperature. 10 ml 1N HCl are added and the organic layer is extracted three times with dichloromethane. The collected extracts are evaporated to give 142 mg (99.9%) of product

¹H-NMR (400 MHz, D₆-DMSO): δ=10.26 (s, 1H, NH), 8.83(s, 1H, oxazole), 8.11(s, 1H, triazole), 7.70(s, 1H, triazole), 7.57 (d, 2H, 3,5-H-2,6-Cl-phenyl), 7.39 (m, 1H, 4-H-2,6-Cl-phenyl) 7.13 (d, 2H, Ar—H, phenoxy), 6.98 (d, 2H, Ar—H, phenoxy), 5.28 (s, 2H, CH₂—O-Ph), 4.39 (t, 2H, 1H-butyl), 2.54 (t, 2H, 4H-butyl), 1.81 (m, 2H, 2H-butyl), 1.48 (m, 2H, 3H-butyl).

MS: M=485.9 (API+)

### EXAMPLE 45

### 2-[4-(4-[1,2,3]Triazol-1-yl-butyl)-phenoxymethyl]-oxazole-4-carboxylic acid (2,4-dichloro-phenyl)-amide

The title compound is prepared from 100 mg (0.29 mmol) 2-[4-(4-[1,2,3]Triazol-1-yl-butyl)-phenoxymethyl]-oxazole-4-carboxylic acid and 52 mg (0.32 mmol) 2,4-dichloro-phenylamine as described in example 1. Purification of the product is achieved by preparative HPLC. Yield: 3 mg (2%).

MS: M=486.3 (API+)

### EXAMPLE 46

### 2-[3-Methyl-4-(4-[1,2,3]triazol-1-yl-butyl)-phenoxymethyl]-oxazole-4-carboxylic acid (4-trifluoromethyl-phenyl)-amide

170 mg (0.48 mmol) 2-[3-Methyl-4-(4-[1,2,3]triazol-1-yl-butyl)-phenoxymethyl]-oxazole-4-carboxylic acid are dissolved in 0.2 ml dichloromethane and activated with 0.4 ml (5.51 mmol) thionyl chloride. The mixture is heated to 45° C. and stirred for 1 hour. After evaporation to dryness the residue is dissolved in 2 ml dichloromethane. 77.0 mg (0.48 mmol) 4-trifluoromethyl-phenyl)-amine and 0.33 ml (2.39 mmol) triethylamine are added. The resulting mixture is stirred for 2 h at room temperature. After the addition of 1N hydrochloric acid the mixture is extracted twice with dichloromethane. The organic layer is evaporated and the residue is purified by silica column flash chromatography with ethyl acetate/heptane (1:1) to give the title compound. Yield: 69 mg (29%).

¹H-NMR (400 MHz, D₆-DMSO): δ=10.59 (s, 1H, NH), 8.88 (s, 1H, 5-H oxazole), 8.11 (s, 1H, triazole), 8.05 (m, 2H, Ar—H, Ph-CF₃), 7.72 (m, 2H, Ar—H, Ph-CF₃), 7.70(s, 1H, triazole), 7.03 (m, 1H, Ar—H, phenoxy-methyl), 6.85 (m, 1H, Ar—H, phenoxy-methyl), 6.80 (m, 1H, Ar—H, phenoxy3-methyl), 5.26 (s, 2H, CH₂—O-Ph), 4.40 (t, 2H, 1H-butyl), 2.54 (t, 2H, 4H-butyl), 2.21 (s, 3H, Ph-CH₃), 1.86 (m, 2H, 2H-butyl), 1.42 (m, 2H, 3H-butyl).

MS: M=500.4 (API+)

### EXAMPLE 47

### 2-[3-Methyl-4-(4-[1,2,3]triazol-1-yl-butyl)-phenoxymethyl]-oxazole-4-carboxylic acid (4-chloro-phenyl)-amide

The title compound is prepared from 168 mg (0.47 mmol) 2-[3-Methyl-4-(4-[1,2,3]triazol-1-yl-butyl)-phenoxymethyl]-oxazole-4-carboxylic acid and 60.0 mg (0.47 mmol) 4-chloro-phenylamine as described in example 46. Purification of the product is achieved by preparative HPLC. Yield: 80 mg (37%).

¹H-NMR (400 MHz, D₆-DMSO): δ=10.37 (s, 1H, NH), 8.83 (s, 1H, 5-H oxazole), 8.11 (s, 1H, triazole), 7.85 (m, 2H, Ar—H, Ph—Cl), 7.72 (s, 1H, triazole), 7.40 (m, 2H, Ar—H, Ph-Cl), 7.03 (m, 1H, Ar—H, phenoxy-methyl), 6.84 (m, 1H, Ar—H, phenoxy-methyl), 6.80 (m, 1H, Ar—H, phenoxy-methyl), 5.25 (s, 2H, CH₂—O-Ph), 4.40 (t, 2H, 1H-butyl), 2.53 (t, 2H, 4H-butyl), 2.21 (s, 3H, Ph—CH₃), 1.86 (m, 2H, 2H-butyl), 1.42 (m, 2H, 3H-butyl).

MS: M 466.3(API+)

### EXAMPLE 48

### 2-[3-Methyl-4-(4-[1,2,3]triazol-1-yl-butyl)-phenoxymethyl]-oxazole-4-carboxylic acid (3-chloro-phenyl)-amide

The title compound is prepared from 196 mg (0.55 mmol) 2-[3-Methyl-4-(4-[1,2,3]triazol-1-yl-butyl)-phenoxymethyl]-oxazole-4-carboxylic acid and 70.0 mg (0.55 mmol) 3-chloro-phenylamine as described in example. 46. Yield: 137 mg (54%).

¹H-NMR (400 MHz, D₆-DMSO): δ=10.42 (s, 1H, NH), 8.84 (s, 1H, 5-H oxazole), 8.11 (s, 1H, triazole), 7.99 (m, 1H, Ar—H, 3-Cl-Ph), 7.76 (m, 1H, Ar—H, 3-Cl-Ph), 7.70 (s, 1H, triazole), 7.37 (m, 1H, Ar—H, 3-Cl-Ph), 7.17 (m, 1H, Ar—H, 3-Cl-Ph), 7.03 (m, 1H, Ar—H, phenoxy-methyl), 6.85 (m, 1H, Ar—H, phenoxy-methyl), 6.80 (m, 1H, Ar—H, phenoxy-methyl), 5.25 (s, 2H, CH₂—O—Ph), 4.40 (t, 2H, 1H-butyl), 2.53 (t, 2H, 4H-butyl), 2.21 (s, 3H, Ph-CH₃), 1.86 (m, 2H, 2H-butyl), 1.42 (m, 2H, 3H-butyl).

MS: M=466.3 (API+)

### EXAMPLE 49

### 2-[3-Methyl-4-(4-[1,2,3]triazol-1-yl-butyl)-phenoxymethyl]-thiazole-4-carboxylic acid (4-trifluoromethyl-phenyl)-amide

The title compound is prepared from 178 mg (0.48 mmol) 2-[3-Methyl-4-(4-[1,2,3]triazol-1-yl-butyl)-phenoxymethyl]-oxazole-4-carboxylic acid and 60.0 mg (0.55 mmol) 4-trifluoromethyl-phenylamine as described in example 46. Purification of the product is achieved by preparative HPLC. Yield: 145 mg (59%).

¹H-NMR (400 MHz, D₆-DMSO): δ=10.67 (s, 1H, NH), 8.53 (s, 1H, 5-H thiazole), 8.11 (s, 1H, triazole), 8.09 (m, 2H, Ar—H, Ph-CF₃), 7.73 (m, 2H, Ar—H, Ph-CF₃), 7.71 (s, 1H, triazole), 7.05 (m, 1H, Ar—H, phenoxy-methyl), 6.89 (m, 1H, Ar—H, phenoxy-methyl), 6.83 (m, 1H, Ar—H, phenoxy-methyl), 5.47 (s, 2H, CH₂—O-Ph), 4.41 (t, 2H, 1H-butyl), 2.54 (t, 2H, 4H-butyl), 2.22 (s, 3H, Ph-CH₃), 1.86 (m, 2H, 2H-butyl), 1.43 (m, 2H, 3H-butyl).

MS: M=516.3 (API+)

### EXAMPLE 50

### 2-[3-Methyl-4-(4-[1,2,3]triazol-1-yl-butyl)-phenoxymethyl]-thiazole-4-carboxylic acid (4-chloro-phenyl)-amide

The title compound is prepared from 204 mg (0.55 mmol) 2-[3-Methyl-4-(4-[1,2,3]triazol-1-yl-butyl)-phenoxymethyl]-thiazole-4-carboxylic acid and 70.0 mg (0.55 mmol) 4-chloro-phenylamine as described in example 46. Purification of the product is achieved by preparative HPLC. Yield: 55 mg (21%).

¹H-NMR (400 MHz, D₆-DMSO): δ=10.44 (s, 1H, NH), 8.47 (s, 1H, 5-H thiazole), 8.11 (s, 1H, triazole), 7.88 (m, 2H, Ar—H, Ph-Cl), 7.70 (s, 1H, triazole), 7.41 (m, 2H, Ar—H, Ph-Cl), 7.04 (m, 1H, Ar—H, phenoxy-methyl), 6.88 (m, 1H, Ar—H, phenoxy-methyl), 6.83 (m, 1H, Ar—H, phenoxy-methyl), 5.46 (s, 2H, CH₂—O-Ph), 4.41 (t, 2H, 1H-butyl), 2.54 (t, 2H, 4H-butyl), 2.22 (s, 3H, Ph—CH₃), 1.86 (m, 2H, 2H-butyl), 1.43 (m, 2H, 3H-butyl).

MS: M=482.1 (API+)

### EXAMPLE 51

### 2-[3-Methyl-4-(4-[1,2,3]triazol-1-yl-butyl)-phenoxymethyl]-thiazole-4-carboxylic acid (3-chloro-phenyl)-amide

The title compound is prepared from 204 mg (0.55 mmol) 2-[3-Methyl-4-(4-[1,2,3]triazol-1-yl-butyl)-phenoxymethyl]-thiazole-4-carboxylic acid and 70.0 mg (0.55 mmol) 3-chloro-phenylamine as described in example 46. Purification of the product is achieved by preparative HPLC. Yield: 77 mg (29%).

¹H-NMR (400 MHz, D₆-DMSO): δ=10.49 (s, 1H, NH), 8.49 (s, 1H, 5-H thiazole), 8.11 (s, 1H, triazole), 8.04 (m, 1H, Ar—H, Ph-Cl), 7.79 (m, 1H, Ar—H, Ph-Cl), 7.70 (s, 1H, triazole), 7.38 (m, 1H, Ar—H, Ph-Cl), 7.17 (m, 1H, Ar—H, Ph-Cl), 7.04 (m, 1H, Ar—H, phenoxy-methyl), 6.88 (m, 1H, Ar—H, phenoxy-methyl), 6.83 (m, 1H, Ar—H, phenoxy-methyl), 5.46 (s, 2H, CH₂—O-Ph), 4.41 (t, 2H, 1H-butyl), 2.54 (t, 2H, 4H-butyl), 2.21 (s, 3H, Ph-CH₃), 1.86 (m, 2H, 2H-butyl), 1.43 (m, 2H, 3H-butyl).

MS: M=482.2 (API+)

### EXAMPLE 52-1

### 4-Nitro-N-{4-[4-(4-[1,2,3]triazol-1-yl-butyl)-phenoxymethyl]-thiazol-2-yl}-benzamide Preparation of N-(4-Chloromethyl-thiazol-2-yl)-acetamide

A solution of 50 g (0.42 mol) acetyl thiourea and 108.5 g 1,3-dichloro-propan-2-one (0.84 mol) in 500 ml acetone is heated to reflux for 5 hours. All volatiles were removed in vacuo and the residue was taken up in 200 ml acetone. After addition of 600 ml of water the precipitate was collected, washed with 250 ml n-heptane and dried in vacuo at 40° C. Yield: 56.9 g (71%) white solid.

¹H-NMR (400 MHz, D₆-DMSO): δ=2.13 (s, 3H); 4.71 (s, 2H); 7.22 (s, 1H); 12.21 (s, 1H)

### Preparation of N-{4-[4-(4-[1,2,3]Triazol-1-yl-butyl)-phenoxymethyl]-thiazol-2-yl}-acetamide

To a solution of 8.55 g (39 mmol) 4-(4-[1,2,3]Triazol-1-yl-butyl)-phenol in 150 ml 2-butanone 12.81 g (39 mmol) cesium carbonate were added and the mixture was stirred at 80° C. for 30 minutes. Then 3.75 g (20 mmol) N-(4-Chloromethyl-thiazol-2-yl)-acetamide and 5.90 g (39 mmol) sodium iodide were added and the mixture was stirred at 60° C. overnight. After concentration in vacuo 50 ml of a saturated aqueous solution of sodium chloride was added and the mixture was extracted with a mixture of CH₂Cl₂ and methanol (5:1, 60 ml each time). The combined organic phases were dried over Na₂SO₄ and evaporated to dryness. Purification by preparative scale HPLC (RP 18, methanol-water-gradient) returned 2.08 g (47%) of the title compound and 5.97 g unreacted 4-(4-[1,2,3]-triazol-1-yl-butyl)-phenol.

¹H-NMR (400 MHz, D₆-DMSO): δ=1.47 (quintet, 2H); 1.81 (quintet 2H); 2.13 (s, 3H); 2.52 (t, 2H); 4.39 (t, 2H); 5.00 (s, 2H); 6.91 (d, 2H); 7.07 (d, 2H); 7.16 (s, 1H); 7.70 (s, 1H); 8.10 (s, 1H); 12.15 (s, 1H)

### Preparation of 4-[4-(4-[1,2,3]Triazol-1-yl-butyl)-phenoxymethyl]-thiazol-2-ylamine

To a solution of 2.05 g (5.5 mmol) N-{4-[4-(4-[1,2,3]Triazol-1-yl-butyl)-phenoxymethyl]-thiazol-2-yl}-acetamide in 60 ml methanol a solution of 396 mg (16.6 mmol) lithium hydroxide in 60 ml water was added and the mixture was stirred at 60° C. overnight. After evaporation to dryness 20 ml of a saturated aqueous solution of sodium chloride was added and the pH was adjusted to 3 with 1 N HCl. The mixture was washed twice with ethyl acetate (discarded) and then the pH was adjusted to 9 with aqueous NaOH (30%). Subsequent extraction with a mixture of CH₂Cl₂ and methanol (5:1, three times 50), drying of the combined organic layers over Na₂SO₄ and evaporation to dryness yielded 1.55 g (85%) of the title compound.

¹H-NMR (400 MHz, D₆-DMSO): δ=1.47 (quintet, 2H); 1.80 (quintet 2H); 2.52 (t, 2H); 4.39 (t, 2H); 4.79 (s, 2H); 6.53 (s, 1H); 6.88 (d, 2H); 6.97 (s, 2H); 7.06 (d, 2H); 7.70 (s, 1H); 8.13 (s, 1H)

### Preparation of 4-Nitro-N-{4-[4-(4-[1,2,3]triazol-1-yl-butyl)-phenoxymethyl]-thiazol-2-yl}-benzamide

4-Nitrobenzoyl chloride (84 mg, 0.45 mmol) was added to a solution of 4-[4-(4-[1,2,3]Triazol-1-yl-butyl)-phenoxymethyl]-thiazol-2-ylamine (50 mg, 0.15 mmol) in DCM (2 ml). Polymer supported methylpiperidine (150 mg, 0.45 mmol) was then added to the reaction mixture and the reaction mixture was shaken for 48 hours at room temperature. AMPS (aminomethylpolystyrene) (200 mg, 0.45 mmol) was then added and the reaction mixture was shaken for a further 16 hours. The reaction mixture was filtered and the resin was washed with methanol (5 ml). The organics were combined and the solvent was removed under reduced pressure. The crude product was purified by preparative HPLC under neutral conditions to give 4-Nitro-N-{4-[4-(4-[1,2,3]triazol-1-yl-butyl)-phenoxymethyl]-thiazol-2-yl}-benzamide, 31.1 mg (46% yield).

MS(ESI+)

¹H-NMR (400 MHz, D₆-DMSO): δ=1.48 (quintet, 2H); 1.81 (quintet 2H); 2.53 (t, 2H); 4.39 (t, 2H); 5.07 (s, 2H); 6.93 (s, 1H); 7.09 (d, 2H); 7.35 (s, 1H); 7.70 (s, 1H); 8.11 (s, 1H); 8.31 (d, 2H); 8.37 (d, 2H); 13.10 (s, 1H)

The following examples are prepared in an analogous manner to Example 52-1 starting from the appropriate materials:

| Example | | |
|---|---|---|
| No. | Systematic name | MS (Method) |
| 52-2 | N-{4-[4-(4-[1,2,3]Triazol-1-yl-butyl)- | 502.3 (ESI+) |
| | phenoxymethyl]-thiazol-2-yl}-3- | |
| | trifluoromethyl-benzamide | |
| 52-3 | 2,4-Difluoro-N-{4-[4-(4-[1,2,3]triazol- | 470.2 (ESI+) |
| | 1-yl-butyl)-phenoxymethyl]-thiazol-2- | |
| | yl}-benzamide | |
| 52-4 | N-{4-[4-(4-[1,2,3]Triazol-1-yl-butyl)- | 518.3 (ESI+) |
| | phenoxymethyl]-thiazol-2-yl}-4- | |
| | trifluoromethoxy-benzamide | |
| 52-5 | 4-Chloro-N-{4-[4-(4-[1,2,3]triazol-1- | 468.2 (ESI+) |
| | yl-butyl)-phenoxymethyl]-thiazol-2-yl}- | |
| | benzamide | |
| 52-6 | 4-Chloro-3-fluoro-N-{4-[4-(4- | 486.1 (ESI+) |
| | [1,2,3]triazol-1-yl-butyl)- | |
| | phenoxymethyl]-thiazol-2-yl}- | |
| | benzamide | |
| 52-7 | N-{4-[4-(4-[1,2,3]Triazol-1-yl-butyl)- | 534.3 (ESI+) |
| | phenoxymethyl]-thiazol-2-yl}-4- | |
| | trifluoromethylsulfanyl-benzamide | |
| 52-8 | 4-Cyano-N-{4-[4-(4-[1,2,3]triazol-1-yl- | 459.0 (ESI+) |
| | butyl)-phenoxymethyl]-thiazol-2-yl}- | |
| | benzamide | |
| 52-9 | 3,4-Dichloro-N-{4-[4-(4-[1,2,3]triazol- | 502.2 (ESI+) |
| | 1-yl-butyl)-phenoxymethyl]-thiazol-2- | |
| | yl}-benzamide | |
| 52-10 | 2-Fluoro-N-{4-[4-(4-[1,2,3]triazol-1-yl- | 520.2 (ESI+) |
| | butyl)-phenoxymethyl]-thiazol-2-yl}-4- | |
| | trifluoromethyl-benzamide | |
| 52-11 | 2,2-Difluoro-benzo[1,3]dioxole-5- | 514.0 (ESI+) |
| | carboxylic acid{4-[4-(4-[1,2,3]triazol- | |
| | 1-yl-butyl)-phenoxymethyl]-thiazol-2- | |
| | yl}-amide | |
| 52-12 | 3-Chloro-N-{4-[4-(4-[1,2,3]triazol-1- | 468.2 (ESI+) |
| | yl-butyl)-phenoxymethyl]-thiazol-2-yl}- | |
| | benzamide | |
| 52-13 | 4-Chloro-2-fluoro-N-{4-[4-(4- | 486.0 (ESI+) |
| | [1,2,3]triazol-1-yl-butyl)- | |
| | phenoxymethyl]-thiazol-2-yl}- | |
| | benzamide | |
| 52-14 | 4-Methyl-N-{4-[4-(4-[1,2,3]triazol-1- | 448.4 (ESI+) |
| | yl-butyl)-phenoxymethyl]-thiazol-2-yl}- | |
| | benzamide | |
| 52-15 | 2,5-Difluoro-N-{4-[4-(4-[1,2,3]triazol- | 470.3 (ESI+) |
| | 1-yl-butyl)-phenoxymethyl]-thiazol-2- | |
| | yl}-benzamide | |
| 52-16 | 2-Chloro-N-{4-[4-(4-[1,2,3]triazol-1- | 468.3 (ESI+) |
| | yl-butyl)-phenoxymethyl]-thiazol-2-yl}- | |
| | benzamide | |
| 52-17 | 2,3-Difluoro-N-{4-[4-(4-[1,2,3]triazol- | 470.3 (ESI+) |
| | 1-yl-butyl)-phenoxymethyl]-thiazol-2- | |
| | yl}-benzamide | |
| 52-18 | 2-Fluoro-N-{4-[4-(4-[1,2,3]triazol-1-yl- | 520.3 (ESI+) |
| | butyl)-phenoxymethyl]-thiazol-2-yl}-6- | |
| | trifluoromethyl-benzamide | |
| 52-19 | 3-Chloro-4-fluoro-N-{4-[4-(4- | 486.3 (ESI+) |
| | [1,2,3]triazol-1-yl-butyl)- | |
| | phenoxymethyl]-thiazol-2-yl}- | |
| | benzamide | |
| 52-20 | N-{4-[4-(4-[1,2,3]Triazol-1-yl-butyl)- | 502.2 (ESI+) |
| | phenoxymethyl]-thiazol-2-yl}-4- | |
| | trifluoromethyl-benzamide | |
| 52-21 | 4-tert-Butyl-N-{4-[4-(4-[1,2,3]triazol- | 490.2 (ESI+) |
| | 1-yl-butyl)-phenoxymethyl]-thiazol-2- | |
| | yl}-benzamide | |
| 52-22 | 4-Difluoromethoxy-N-{4-[4-(4- | 500.2 (ESI+) |
| | [1,2,3]triazol-1-yl-butyl)- | |
| | phenoxymethyl]-thiazol-2-yl}- | |
| | benzamide | |
| 52-23 | 3,5-Difluoro-N-{4-[4-(4-[1,2,3]triazol- | 470.2 (ESI+) |
| | 1-yl-butyl)-phenoxymethyl]-thiazol-2- | |
| | yl}-benzamide | |
| 52-24 | 2-Fluoro-N-{4-[4-(4-[1,2,3]triazol-1-yl- | 452.1 (ESI+) |
| | butyl)-phenoxymethyl]-thiazol-2-yl}- | |
| | benzamide | |
| 52-25 | 3-Fluoro-N-{4-[4-(4-[1,2,3]triazol-1-yl- | 452.1 (ESI+) |
| | butyl)-phenoxymethyl]-thiazol-2-yl}- | |
| | benzamide | |
| 52-26 | 4-Fluoro-3-methyl-N-{4-[4-(4- | 466.2 (ESI+) |
| | [1,2,3]triazol-1-yl-butyl)- | |
| | phenoxymethyl]-thiazol-2-yl}- | |
| | benzamide | |

Unless stated to the contrary, all compounds in the examples were prepared and characterized as described. All ranges recited herein encompass all combinations and subcombinations included within that range limit. All patents and publications cited herein are hereby incorporated by reference in their entirety.

1. A compound according to formula I or pharmaceutically acceptable salts thereof wherein formula I is: 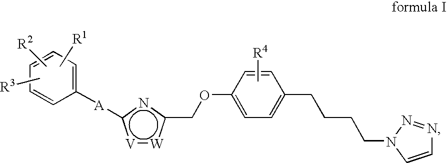
wherein:
R¹ and R² are adjacent and together with the carbon atoms of the phenyl ring to which they are attached form a 5 or 6 membered heterocyclic ring; or alternatively R² is hydrogen and R¹ is selected from the group consisting of:
(a) hydrogen,
(b) halogen,
(c) nitro,
(d) —SF₅,
(e) —O-alkyl, wherein the alkyl group is optionally substituted with one or more halogens;
(f) —S(O)ₙ-alkyl, wherein n is 0, 1 or 2 and wherein the alkyl group is optionally substituted with one or more halogens;
(g) —S(O)₂NH₂,
(h) —S(O)₂NH-acyl,
(i) —S(O)₂NH-heteroaryl,
(j) —NH-alkyl, wherein the alkyl group is optionally substituted with one or more halogens; and
(k) alkyl which is optionally substituted with one or more halogens;
R³ is hydrogen, halogen or nitro;
R⁴ is hydrogen or alkyl;
A is —NHC(O)—, —C(O)NH—, —N(alkyl)C(O)— or —C(O)N(alkyl)-; and
V is —S— and W is —CH—, or alternatively V is —CH— and W is —S— or —O—.

2. The compounds according to claim 1, wherein R⁴ is hydrogen.

3. The compounds according to claim 1, wherein:
R¹ is selected from the group consisting of:
(a) chlorine;
(b) —O-alkyl, wherein the alkyl group is optionally substituted with one or more fluorines;
(c) —S-alkyl, wherein the alkyl group is optionally substituted with one or more fluorines; and
(d) alkyl, which is optionally substituted with one or more fluorines;
R² is hydrogen; and
R³ is hydrogen or fluorine.

4. The compounds according to claim 1, wherein:
A is —NHC(O)— or —N(alkyl)C(O)—.

5. The compounds according to claim 1, wherein:
A is —NHC(O)— or —N(alkyl)C(O)—;
V is —CH—; and
W is —S— or —O—.

6. The compounds according to claim 5, wherein:
R¹ is selected from the group consisting of:
(a) chlorine,
(b) —O—CF₃,
(c) —O—CF₂,
(d) —S—CF₃,
(e) —S—CF₂,
(f) —CF₃, and
(g) —SF₅;
R² is hydrogen; and
R³ is hydrogen; fluorine; chlorine or nitro.

7. A compound according to claim 6 selected from the group consisting of:
2-[4-(4-[1,2,3]Triazol-1-yl-butyl)-phenoxymethyl]-oxazole-4-carboxylic acid (4-chloro-phenyl)-methyl-amide;
2-[4-(4-[1,2,3]Triazol-1-yl-butyl)-phenoxymethyl]-oxazole-4-carboxylic acid (2-nitro-4-trifluoromethyl-phenyl)-amide;
2-[4-(4-[1,2,3]Triazol-1-yl-butyl)-phenoxymethyl]-oxazole-4-carboxylic acid (4-chloro-phenyl)-amide;
2-[4-(4-[1,2,3]Triazol-1-yl-butyl)-phenoxymethyl]-oxazole-4-carboxylic acid (4-difluoromethylsulfanyl-phenyl)-amide;
2-[4-(4-[1,2,3]Triazol-1-yl-butyl)-phenoxymethyl]-oxazole-4-carboxylic acid (4-trifluoro-methoxy-phenyl)-amide;
2-[4-(4-[1,2,3]Triazol-1-yl-butyl)-phenoxymethyl]-oxazole-4-carboxylic acid (4-trifluoro-methylsulfanyl-phenyl)-amide;
2-[4-(4-[1,2,3]Triazol-1-yl-butyl)-phenoxymethyl]-oxazole-4-carboxylic acid (3-chloro-4-fluoro-phenyl)-amide;
2-[4-(4-[1,2,3]Triazol-1-yl-butyl)-phenoxymethyl]-oxazole-4-carboxylic acid (4-trifluoromethyl-phenyl)-amide;
2-[4-(4-[1,2,3]Triazol-1-yl-butyl)-phenoxymethyl]-oxazole-4-carboxylic acid (4-chloro-2-fluoro-phenyl)-amide; and
2-[4-(4-[1,2,3]Triazol-1-yl-butyl)-phenoxymethyl]-oxazole-4-carboxylic acid (4-difluoromethoxy-phenyl)-amide.

8. A compound according to claim 6 selected from the group consisting of:
2-[4-(4-[1,2,3]Triazol-1-yl-butyl)-phenoxymethyl]-oxazole-4-carboxylic acid (3-trifluoromethyl-phenyl)-amide;
2-[4-(4-[1,2,3]Triazol-1-yl-butyl)-phenoxymethyl]-oxazole-4-carboxylic acid (4-chloro-3-trifluoromethyl-phenyl)-amide;
2-[4-(4-[1,2,3] Triazol-1-yl-butyl)-phenoxymethyl]-oxazole-4-carboxylic acid methyl-(4-trifluoromethoxy-phenyl)-amide;
2-[4-(4-[1,2,3]Triazol-1-yl-butyl)-phenoxymethyl]-oxazole-4-carboxylic acid (4-nitro-2-trifluoromethyl-phenyl)-amide;
2-[4-(4-[1,2,3]Triazol-1-yl-butyl)-phenoxymethyl]-thiazole-4-carboxylic acid (4-trifluoromethyl-phenyl)-amide;
2-[4-(4-[1,2,3] Triazol-1-yl-butyl)-phenoxymethyl]-thiazole-4-carboxylic acid (4-chloro-phenyl)-amide;
2-[4-(4-[1,2,3]Triazol-1-yl-butyl)-phenoxymethyl]-thiazole-4-carboxylic acid (4-trifluoromethoxy-phenyl)-amide;
2-[4-(4-[1,2,3]Triazol-1-yl-butyl)-phenoxymethyl]-oxazole-4-carboxylic acid (2,6-dichloro-phenyl)-amide; and
2-[4-(4-[1,2,3]Triazol-1-yl-butyl)-phenoxymethyl]-oxazole-4-carboxylic acid (2,4-dichloro-phenyl)-amide.

9. The compounds according to claim 5 wherein R³ is hydrogen, fluorine or nitro; and R¹ and R² are adjacent and together with the carbon atoms of the phenyl ring to which they are attached form a 2,2-difluoro-benzo[1,3]dioxolyl moiety or a benzo[1,3]dioxolyl moiety; or alternatively, R² is hydrogen and R¹ is selected from the group consisting of:
(a) hydrogen,
(b) fluorine,
(c) bromine,
(d) —O-alkyl,
(e) —S-alkyl,
(f) alkyl,
(g) —S(O)₂NH₂,
(h) —S(O)₂NH-acyl, and
(i) —S(O)₂NH-heteroaryl.

10. A compound according to claim 9 selected from the group consisting of:
2-[4-(4-[1,2,3]Triazol-1-yl-butyl)-phenoxymethyl]-oxazole-4-carboxylic acid benzo[1,3]dioxol-5-yl-amide;
2-[4-(4-[1,2,3]Triazol-1-yl-butyl)-phenoxymethyl]-oxazole-4-carboxylic acid (3,5-difluoro-phenyl)-amide;
2-[4-(4-[1,2,3]Triazol-1-yl-butyl)-phenoxymethyl]-oxazole-4-carboxylic acid (2,4-difluoro-phenyl)-amide;
2-[4-(4-[1,2,3]Triazol-1-yl-butyl)-phenoxymethyl]-oxazole-4-carboxylic acid (2,2-difluoro-benzo [1,3] dioxol-5-yl)-amide;
2-[4-(4-[1,2,3]Triazol-1-yl-butyl)-phenoxymethyl]-oxazole-4-carboxylic acid (4-(thiazol-2-ylsulfamoyl)-phenyl)-amide;
2-[4-(4-[1,2,3]Triazol-1-yl-butyl)-phenoxymethyl]-oxazole-4-carboxylic acid [4-(5-methyl-[1,3,4] thiadiazol-2-ylsulfamoyl)-phenyl]-amide;
2-[4-(4-[1,2,3]Triazol-1-yl-butyl)-phenoxymethyl]-oxazole-4-carboxylic acid [4-(5-methyl-isoxazol-3-ylsulfamoyl)-phenyl]-amide;
2-[4-(4-[1,2,3]Triazol-1-yl-butyl)-phenoxymethyl]-oxazole-4-carboxylic acid (4-acetylsulfamoyl-phenyl)-amide;
2-[4-(4-[1,2,3]Triazol-1-yl-butyl)-phenoxymethyl]-oxazole-4-carboxylic acid (4-bromo-2-fluoro-phenyl)-amide; and
2-[4-(4-[1,2,3]Triazol-1-yl-butyl)-phenoxymethyl]-oxazole-4-carboxylic acid (3-fluoro-2-methyl-phenyl)-amide.

11. A compound according to claim 9 selected from the group consisting of:
2-[4-(4-[1,2,3]Triazol-1-yl-butyl)-phenoxymethyl]-oxazole-4-carboxylic acid (4-fluoro-3-nitro-phenyl)-amide;
2-[4-(4-[1,2,3]Triazol-1-yl-butyl)-phenoxymethyl]-oxazole-4-carboxylic acid (4-methylsulfanyl-phenyl)-amide;
2-[4-(4-[1,2,3]Triazol-1-yl-butyl)-phenoxymethyl]-oxazole-4-carboxylic acid (4-fluoro-2-methyl-phenyl)-amide;
2-[4-(4-[1,2,3]Triazol-1-yl-butyl)-phenoxymethyl]-oxazole-4-carboxylic acid (4-tert-butyl-phenyl)-amide;
2-[4-(4-[1,2,3]Triazol-1-yl-butyl)-phenoxymethyl]-oxazole-4-carboxylic acid p-tolylamide;
2-[4-(4-[1,2,3]Triazol-1-yl-butyl)-phenoxymethyl]-oxazole-4-carboxylic acid methyl-phenyl-amide; and
2-[4-(4-[1,2,3]Triazol-1-yl-butyl)-phenoxymethyl]-oxazole-4-carboxylic acid (4-fluoro-phenyl)-amide.

12. A compound according to claim 9 selected from the group consisting of:
2-[4-(4-[1,2,3]Triazol-1-yl-butyl)-phenoxymethyl]-oxazole-4-carboxylic acid (4-fluoro-3-methyl-phenyl)-amide;
2-[4-(4-[1,2,3]Triazol-1-yl-butyl)-phenoxymethyl]-oxazole-4-carboxylic acid (2-fluoro-phenyl)-amide;
2-[4-(4-[1,2,3]Triazol-1-yl-butyl)-phenoxymethyl]-oxazole-4-carboxylic acid (3-fluoro-phenyl)-amide;
2-[4-(4-[1,2,3]Triazol-1-yl-butyl)-phenoxymethyl]-oxazole-4-carboxylic acid (3-fluoro-4-methyl-phenyl)-amide;
2-[4-(4-[1,2,3]Triazol-1-yl-butyl)-phenoxymethyl]-oxazole-4-carboxylic acid (4-methoxy-phenyl)-amide; and
2-[4-(4-[1,2,3]Triazol-1-yl-butyl)-phenoxymethyl]-oxazole-4-carboxylic acid (3,4-difluoro-phenyl)-amide.

13. The compounds according to claim 1, wherein:
A is —NHC(O)— or —N(alkyl)C(O)—;
V is —S—; and
W is —CH—.

14. The compounds according to claim 13, wherein:
R¹ is selected from the group consisting of:
(a) chlorine;
(b) —O-alkyl, wherein the alkyl group is optionally substituted with one or more fluorines;
(c) —SF₅; and
(d) alkyl, which is optionally substituted with one or more fluorines;
R² is hydrogen; and
R³ is hydrogen or fluorine.

15. A compound according to claim 14 selected from the group consisting of:
4-[4-(4-[1,2,3]Triazol-1-yl-butyl)-phenoxymethyl]-thiazole-2-carboxylic acid (4-chloro-phenyl)-amide;
4-[4-(4-[1,2,3]Triazol-1-yl-butyl)-phenoxymethyl]-thiazole-2-carboxylic acid (4-trifluoromethoxy-phenyl)-amide; and
4-[4-(4-[1,2,3]Triazol-1-yl-butyl)-phenoxymethyl]-thiazole-2-carboxylic acid (4-pentafluorosulfanyl-phenyl)-amide.

16. The compounds according to claim 1, wherein:
A is —C(O)NH— or —C(O)N(alkyl)-.

17. The compounds according to claim 1, wherein:
A is —C(O)NH— or —C(O)N(alkyl)-;
V is —S—; and
W is —CH—.

18. The compounds according to claim 14, wherein:
R¹ is selected from the group consisting of:
(a) chlorine;
(b) —O-alkyl, wherein the alkyl group is optionally substituted with one or more fluorines; and
(c) alkyl, wherein the alkyl group is optionally substituted with one or more fluorines;
R² is hydrogen; and
R³ is hydrogen, chlorine or fluorine.

19. A compound according to claim 18 selected from the group consisting of:
N-{4-[4-(4-[1,2,3]Triazol-1-yl-butyl)-phenoxymethyl]-thiazol-2-yl}-3-trifluoromethyl-benzamide;
N-{4-[4-(4-[1,2,3]Triazol-1-yl-butyl)-phenoxymethyl]-thiazol-2-yl}-4-trifluoromethoxy-benzamide;
4-Chloro-N-{4-[4-(4-[1,2,3]triazol-1-yl-butyl)-phenoxymethyl]-thiazol-2-yl}-benzamide;
4-Chloro-3-fluoro-N-{4-[4-(4-[1,2,3]triazol-1-yl-butyl)-phenoxymethyl]-thiazol-2-yl}-benzamide;
3,4-Dichloro-N-{4-[4-(4-[1,2,3]triazol-1-yl-butyl)-phenoxymethyl]-thiazol-2-yl}-benzamide;
2-Fluoro-N-{4-[4-(4-[1,2,3]triazol-1-yl-butyl)-phenoxymethyl]-thiazol-2-yl}-4-trifluoromethyl-benzamide;
3-Chloro-N-{4-[4-(4-[1,2,3]triazol-1-yl-butyl)-phenoxymethyl]-thiazol-2-yl}-benzamide; and
4-Chloro-2-fluoro-N-{4-[4-(4-[1,2,3]triazol-1-yl-butyl)-phenoxymethyl]-thiazol-2-yl}-benzamide.

20. A compound according to claim 18 selected from the group consisting of:
4-Methyl-N-{4-[4-(4-[1,2,3]triazol-1-yl-butyl)-phenoxymethyl]-thiazol-2-yl}-benzamide; and
2-Chloro-N-{4-[4-(4-[1,2,3]triazol-1-yl-butyl)-phenoxymethyl]-thiazol-2-yl}-benzamide;
2-Fluoro-N-{4-[4-(4-[1,2,3]triazol-1-yl-butyl)-phenoxymethyl]-thiazol-2-yl}-6-trifluoromethyl-benzamide;
3-Chloro-4-fluoro-N-{4-[4-(4-[1,2,3]triazol-1-yl-butyl)-phenoxymethyl]-thiazol-2-yl}-benzamide;
N-{4-[4-(4-[1,2,3]Triazol 1-yl-butyl)-phenoxymethyl]-thiazol-2-yl}-4-trifluoromethyl-benzamide;
4-tert-Butyl-N-{4-[4-(4-[1,2,3]triazol-1-yl-butyl)-phenoxymethyl]-thiazol-2-yl}-benzamide; and
4-Difluoromethoxy-N-{4-[4-(4-[1,2,3]triazol-1-yl-butyl)-phenoxymethyl]-thiazol-2-yl}-benzamide.

21. The compounds according to claim 17, wherein:
R¹ is selected from the group consisting of:
(a) nitro;
(b) cyano;
(c) —S-alkyl; wherein the alkyl group is optionally substituted with one or more fluorines;
(d) fluorine; and
(e) alkyl, which is optionally substituted with one or more fluorines;
R² is hydrogen; and
R³ is hydrogen or fluorine.

22. A compound according to claim 21 selected from the group consisting of:
4-Nitro-N-{4-[4-(4-[1,2,3]triazol-1-yl-butyl)-phenoxymethyl]-thiazol-2-yl}-benzamide;
2,4-Difluoro-N-{4-[4-(4-[1,2,3]triazol-1-yl-butyl)-phenoxymethyl]-thiazol-2-yl}-benzamide;
N-{4-[4-(4-[1,2,3]Triazol-1-yl-butyl)-phenoxymethyl]-thiazol-2-yl}-4-trifluoromethylsulfanyl-benzamide;
4-Cyano-N-{4-[4-(4-[1,2,3]triazol-1-yl-butyl)-phenoxymethyl]-thiazol-2-yl}-benzamide;
2,2-Difluoro-benzo[1,3] dioxole-5-carboxylic acid {4-[4-(4-[1,2,3]triazol-1-yl-butyl)-phenoxymethyl]-thiazol-2-yl}-amide;
2,5-Difluoro-N-{4-[4-(4-[1,2,3]triazol-1-yl-butyl)-phenoxymethyl]-thiazol-2-yl}-benzamide;
2,3-Difluoro-N-{4-[4-(4-[1,2,3]triazol-1-yl-butyl)-phenoxymethyl]-thiazol-2-yl}-benzamide;
3,5-Difluoro-N-{4-[4-(4-[1,2,3]triazol-1-yl-butyl)-phenoxymethyl]-thiazol-2-yl}-benzamide;
2-Fluoro-N-{4-[4-(4-[1,2,3]triazol-1-yl-butyl)-phenoxymethyl]-thiazol-2-yl}-benzamide;
3-Fluoro-N-{4-[4-(4-[1,2,3]triazol-1-yl-butyl)-phenoxymethyl]-thiazol-2-yl}-benzamide; and
4-Fluoro-3-methyl-N-{4-[4-(4-[1,2,3]triazol-1-yl-butyl)-phenoxymethyl]-thiazol-2-yl}-benzamide.

23. The compounds according to claim 1, wherein:
A is —C(O)NH— or —C(O)N(alkyl)-;
V is —CH—; and
W is —S— or —O—.

24. The compounds according to claim 1, wherein:
R⁴ is methyl.

25. The compounds according to claim 24, wherein:
R¹ is selected from the group consisting of:
(a) chlorine;
(b) —O-alkyl, wherein the alkyl group is optionally substituted with one or more fluorines;
(c) —S-alkyl, wherein the alkyl group is optionally substituted with one or more fluorines; and
(d) alkyl, which is optionally substituted with one or more fluorines;
R² is hydrogen;
R³ is hydrogen or fluorine.
A is —NHC(O)— or —N(alkyl)C(O)—;
V is —CH—; and
W is —S— or —O—.

26. The compounds according to claim 20, wherein:
R¹ is chlorine, —O—CF₃, or —CF₃;
R³ is hydrogen;
A is —NHC(O)— or —N(alkyl)C(O)—;
V is —CH—; and
W is —S— or —O—.

27. A compound according to claim 26 selected from the group consisting of:
2-[3-Methyl-4-(4-[1,2,3]triazol-1-yl-butyl)-phenoxymethyl]-oxazole-4-carboxylic acid 4-trifluoromethyl-phenyl)-amide;
2-[3-Methyl-4-(4-[1,2,3]triazol-1-yl-butyl)-phenoxymethyl]-oxazole-4-carboxylic acid (4-chloro-phenyl)-amide;
2-[3-Methyl-4-(4-[1,2,3]triazol-1-yl-butyl)-phenoxymethyl]-oxazole-4-carboxylic acid (3-chloro-phenyl)-amide;
2-[3-Methyl-4-(4-[1,2,3]triazol-1-yl-butyl)-phenoxymethyl]-thiazole-4-carboxylic acid (4-trifluoromethyl-phenyl)-amide;
2-[3-Methyl-4-(4-[1,2,3]triazol-1-yl-butyl)-phenoxymethyl]-thiazole-4-carboxylic acid (4-chloro-phenyl)-amide; and
2-[3-Methyl-4-(4-[1,2,3]triazol-1-yl-butyl)-phenoxymethyl]-thiazole-4-carboxylic acid (3-chloro-phenyl)-amide.

28. A process for the manufacture of the compounds of formula I in claim 1, wherein: the compound of formula V 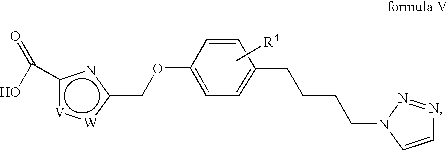
wherein R⁴, V and W are defined according to claim 1, is reacted with a compound of formula VI 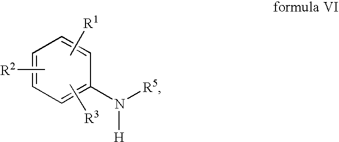
wherein R¹, R², R³ are defined according to claim 1 and R⁵ is hydrogen, to give the respective compound of formula Ia; 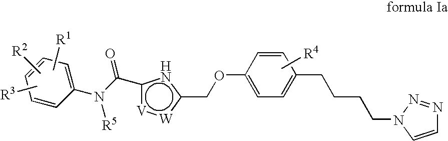
wherein R¹, R², R³, R⁴ are defined according to claim 1 and R⁵ is hydrogen or alkyl.

29. A process according to claim 28, wherein the compound of formula Ia is isolated from the reaction mixture.

30. A process according to claim 28, wherein the compound of formula Ia is converted into a pharmaceutically acceptable salt.

31. A process for the manufacture of the compounds of formula I of claim 1, wherein: the compound of formula IX, 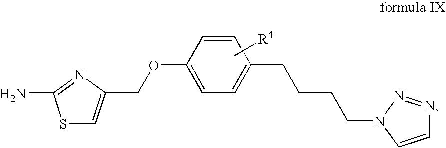
wherein R⁴ is defined according to claim 1, is reacted with a compound of formula X 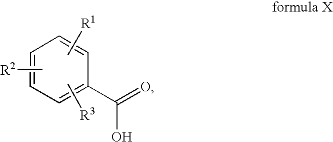
wherein R¹, R², and R³ are defined according to claim 1, to give the respective compound of formula Ib; 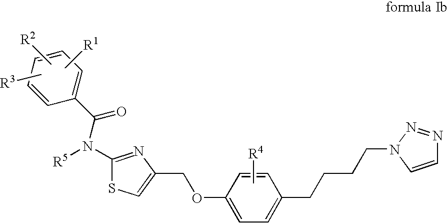
wherein R¹, R², R³ and R⁴ are defined according to claim 1 and R⁵ is hydrogen.

32. A process according to claim 31, wherein the compound of formula Ib is further reacted with a suitable alkyl halide to give the respective compound of formula Ib; wherein R⁵ is alkyl.

33. A process according to claim 31, wherein the compound of formula Ib is isolated from the reaction mixture.

34. A process according to claim 31, wherein the compound of formula Ib is converted into a pharmaceutically acceptable salt.

35. A pharmaceutical composition for preventing or treating cancer or inhibiting tumor growth comprising a therapeutically effective amount of a compound of claim 1 and a pharmaceutically acceptable carrier.

36. A method of preventing or treating a proliferative disease or condition comprising administering to a person in need thereof a therapeutically effective amount of a compound of claim 1.

37. The method of claim 36, wherein the disease or condition is cancer.

38. The method of claim 36, wherein the disease or condition is a proliferating tumor.

39. The method of claim 36, wherein the disease or condition is breast cancer, leukemia, ovarian cancer, lung cancer, pancreatic cancer, or gastrointestinal cancer.
